(19) United States
(12) Patent Application Publication
(10) Pub. No.: US 2025/0205206 A1
(43) Pub. Date: Jun. 26, 2025

(54) USE OF DEXPRAMIPEXOLE FOR THE TREATMENT OF INADEQUATELY CONTROLLED MODERATE TO SEVERE ASTHMA

(71) Applicant: Areteia Therapeutics, Inc., Chapel Hill, NC (US)

(72) Inventors: Eric Bradford, Chapel Hill, NC (US); Calman Philip PRUSSIN, Rockville, MD (US); Gregory C. WILLIAMS, Mendham, NJ (US); Peter WIJNGAARD, Livingston, NC (US); Steven YANCEY, Apex, NC (US)

(73) Assignee: Areteia Therapeutics, Inc., Chapel Hill, NC (US)

(21) Appl. No.: 18/988,479

(22) Filed: Dec. 19, 2024

Related U.S. Application Data
(60) Provisional application No. 63/612,850, filed on Dec. 20, 2023.

(51) Int. Cl. A61K 31/428; A61K 9/20; A61K 9/28; A61K 31/352; A61K 31/4741; A61K 31/522; A61K 31/56; A61P 11/06

(52) U.S. Cl. CPC A61K 31/428; A61K 9/2013; A61K 9/2018; A61K 9/2027; A61K 9/2054; A61K 9/28; A61K 31/352; A61K 31/4741; A61K 31/522; A61K 31/56; A61P 11/06

(57) ABSTRACT

A method of treating inadequately controlled severe asthma of the eosinophilic phenotype in a human subject in need thereof is disclosed. The method comprises orally administering to the subject a daily dose of about 150 mg to about 300 mg of dexpramipexole, or a pharmaceutically acceptable salt thereof, wherein the subject is already receiving at least an inhaled corticosteroid (ICS) and a long-acting beta agonist (LABA), thereby treating the inadequately controlled severe asthma. A method of treating inadequately controlled moderate to severe asthma of the eosinophilic phenotype in a human subject in need thereof is also disclosed. The method comprises orally administering to the subject a daily dose of about 150 mg to about 300 mg of dexpramipexole, or a pharmaceutically acceptable salt thereof, wherein the subject is already receiving at least an ICS and an LABA, thereby treating the inadequately controlled moderate to severe asthma.

### RELATED APPLICATIONS

This application claims priority to U.S. Provisional Application No. 63/612,850 filed Dec. 20, 2023, which is incorporated hereby by reference in its entirety.

### TECHNICAL FIELD

The present disclosure relates generally to methods related to treating inadequately controlled asthma of the eosinophilic phenotype in a human subject in need thereof. More particularly, the present disclose relates to treating inadequately controlled moderate to severe asthma of the eosinophilic phenotype by orally administering dexpramipexole, or a pharmaceutically acceptable salt thereof, to the subject.

### SUMMARY

Embodiments of the present subject matter are directed to methods of treating inadequately controlled severe asthma of the eosinophilic phenotype in a human subject in need thereof comprising orally administering to the subject a daily dose of about 150 mg to about 300 mg of dexpramipexole, or a pharmaceutically acceptable salt thereof, wherein the subject is already receiving at least two asthma medications, thereby treating the inadequately controlled severe asthma of the eosinophilic phenotype in the subject, wherein the at least two asthma medications comprise an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

According to some embodiments, the daily dose of dexpramipexole, or a pharmaceutically acceptable salt thereof, is about 150 mg per day. According to additional embodiments, the daily dose of dexpramipexole, or a pharmaceutically acceptable salt thereof, is administered as 75 mg twice daily.

According to some embodiments, the daily dose of dexpramipexole, or a pharmaceutically acceptable salt thereof, is about 300 mg per day. According to additional embodiments, the daily dose of dexpramipexole, or a pharmaceutically acceptable salt thereof, is administered as 150 mg twice daily.

Embodiments of the present subject matter are directed to methods of treating inadequately controlled moderate to severe asthma of the eosinophilic phenotype in a human subject in need thereof comprising orally administering to the subject a daily dose of about 150 mg to about 300 mg of dexpramipexole, or a pharmaceutically acceptable salt thereof, wherein the subject is already receiving at least two asthma medications, thereby treating the inadequately controlled moderate to severe asthma of the eosinophilic phenotype in the subject, wherein the at least two asthma medications comprise an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

According to some embodiments, the daily dose of dexpramipexole, or a pharmaceutically acceptable salt thereof, is about 150 mg per day. According to additional embodiments, the daily dose of dexpramipexole, or a pharmaceutically acceptable salt thereof, is administered as 75 mg twice daily.

According to some embodiments, the daily dose of dexpramipexole, or a pharmaceutically acceptable salt thereof, is about 300 mg per day. According to additional embodiments, the daily dose of dexpramipexole, or a pharmaceutically acceptable salt thereof, is administered as 150 mg twice daily.

Embodiments of the present subject matter are directed to methods of treating inadequately controlled severe asthma of the eosinophilic phenotype in a human subject in need thereof comprising orally administering to the subject a daily dose of about 150 mg to about 300 mg of dexpramipexole, or a pharmaceutically acceptable salt thereof, wherein orally administering the daily dose reduces a frequency of asthma exacerbations in the subject, thereby treating the inadequately controlled severe asthma of the eosinophilic phenotype in the subject, wherein the subject is already receiving at least two asthma medications comprising an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

According to some embodiments, the frequency of asthma exacerbations is assessed according to an annualized rate of severe asthma exacerbations (AAER).

According to some embodiments, the frequency of asthma exacerbations is assessed according to a time between subsequent severe asthma exacerbations.

### DETAILED DESCRIPTION

This disclosure is not limited to the particular systems, devices and methods described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth. In a further example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg and ranges within, such as 2 mg to 5 mg, 3 mg to 5 mg, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios of a composition are based upon the total weight of the composition and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Dexpramipexole ((6R)-2-amino-4,5,6,7-tetrahydro-6-(propylamino)benzothiazole), is a synthetic aminobenzothiazole derivative with the following structure:

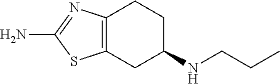

As used herein, dexpramipexole may be administered as a free base or a pharmaceutical acceptable salt, preferably the dihydrochloride salt. As used herein, dexpramipexole is 99.9% to 100% enantiomerically pure. The dexpramipexole contained in a pharmaceutical composition may have a chiral purity for dexpramipexole of at least 99.5%, preferably at least 99.6%, preferably at least 99.7%, preferably at least 99.8%, preferably at least 99.9%, preferably at least 99.95%, or more preferably at least 99.99%. In some embodiments, the chiral purity for dexpramipexole is 100%. In some embodiments, the composition has a chiral purity for dexpramipexole of 99.9% or greater. In some embodiments, the composition has a chiral purity for dexpramipexole of 99.95% or greater. In some embodiments, the composition has a chiral purity for dexpramipexole of 99.99% or greater. The high chiral purity of the pramipexole used herein, dexpramipexole, allows for therapeutic compositions that may have a wide individual and daily dose range.

All embodiments for amounts of, chiral purity of, and dosage form of dexpramipexole, or a pharmaceutically acceptable salt thereof, in the pharmaceutical compositions described herein are provided separately for the sake of brevity, but may be joined in any suitable combination.

Although any methods and materials similar or equivalent to those described herein can be used in the practice or testing of embodiments of the present invention, the exemplary methods, devices, and materials are now described.

In each of the embodiments described herein, the method may consist of orally administering a daily dose of about 150 mg to about 300 mg of dexpramipexole, or a pharmaceutically acceptable salt thereof. Dexpramipexole, or a pharmaceutically acceptable salt thereof, may be administered as 75 mg or 150 mg twice per day. It should be understood that administering the pharmaceutically acceptable salt of dexpramipexole at the recited doses may result in an effective dose of dexpramipexole that is less than recite. In some embodiments, where the pharmaceutically acceptable salt of dexpramipexole is administered at a daily dose of about 150 mg, the effective dose of dexpramipexole may be about 112 mg (e.g., about 56 mg administered twice per day). In some embodiments, where the pharmaceutically acceptable salt of dexpramipexole is administered at a daily dose of about 300 mg, the effective dose of dexpramipexole may be about 224 mg (e.g., about 112 mg administered twice per day).

“Administering” when used in conjunction with a therapeutic means to administer a therapeutic directly or indirectly into or onto a target tissue to administer a therapeutic to a patient whereby the therapeutic positively impacts the tissue to which it is targeted. “Administering” a composition may be accomplished by oral administration in any formulation currently known in the art. “Administering” may include the act of self-administration or administration by another person such as a health care provider.

The term “baseline” refers to the status of the subject prior to the administration of and treatment with dexpramipexole, or pharmaceutically acceptable salt thereof; however, the subject may be receiving other medications for the treatment of asthma.

The term “eosinophil” refers to an eosinophil granulocyte. In some embodiments, the term “eosinophil” refers to a human eosinophil progenitor (hEoP). In some embodiments, the term “eosinophil” refers to an eosinophil lineage-committed progenitor (EoP). In some embodiments, the term “eosinophil” refers to a human common myeloid progenitor (hCMP). In some embodiments, the term “eosinophil” refers to any combination of an eosinophil granulocyte, a human eosinophil progenitor (hEoP), an eosinophil lineage-committed progenitor (EoP), and a human common myeloid progenitor (hCMP). In some embodiments, the term “eosinophil” refers to a CD34+CD125+ progenitor cell. In some embodiments, the term “eosinophil” refers to an eosinophil residing in the bone marrow, in the systemic circulatory system, and/or in organ tissues, including the bone marrow, lungs, and airways. In some embodiments, the organ tissue is the lung, the skin, the heart, the brain, the eye, the gastrointestinal tract, the thymus, the spleen, the kidney, the bladder, the ear, the nose, the sinuses, the oral cavity, the upper respiratory tract, the bone marrow, or combinations thereof.

The term “improves” is used to convey that the present invention changes either the form, characteristics, structure, function and/or physical attributes of the tissue to which it is being provided, applied or administered. “Improves” may also refer to the overall physical state of an individual to whom an active agent has been administered. For example, the overall physical state of an individual may “improve” if one or more symptoms of the disease, condition or disorder are alleviated by administration of an active agent.

In each of the embodiments disclosed herein, the compounds and methods may be utilized with or on a subject in need of such treatment, which may also be referred to as “in need thereof.” The phrase “in need thereof” means that the subject has been identified as having a need for the particular method or treatment and that the treatment is being given to the subject for that particular purpose.

Any reference to symptoms, signs, or biomarkers are herein collectively referred to as “symptom.”

“Optional” or “optionally” may be taken to mean that the subsequently described structure, event or circumstance may or may not occur, and that the described includes instances where the event occurs and instances where it does not.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “therapeutic” means an agent utilized to treat, combat, ameliorate, or prevent an unwanted disease, condition, or disorder of a patient.

The terms “therapeutically effective amount” or “therapeutic dose” is used herein are interchangeable and may refer to the amount of an active agent, pharmaceutical compound, or composition that elicits a clinical, biological, or medicinal response in a tissue, system, animal, individual, or human that is being sought by a researcher, veterinarian, medical doctor, or other clinical professional. A clinical, biological, or medical response may include, for example, inhibiting a disease, condition, or disorder in an individual that is experiencing or displaying the pathology or symptoms of the disease, condition, or disorder, or arresting further development of the pathology and/or symptoms of the disease, condition, or disorder, or ameliorating a disease, condition, or disorder in an individual that is experiencing or exhibiting the pathology or symptoms of the disease, condition, or disorder or reversing the pathology and/or symptoms experience or exhibited by the individual.

The term “prevent” may be taken to mean prophylaxis of a specific disorder, disease, or condition. In some embodiments, the term refers to preventing a disease, condition or disorder in an individual that may be predisposed to the disease, condition or disorder but does not yet experience or display pathology or symptoms of the disease, condition or disorder,

The term “treating” may be taken to mean alleviation of the symptoms associated with a specific disorder, disease, or condition and/or prevention of the worsening of symptoms associated with a specific disorder, disease, or condition. In some embodiments, the term refers to slowing or arresting the progression of the disorder, disease, or condition. In some embodiments, the term refers to alleviating the symptoms associated with the specific disorder, disease, or condition. In some embodiments, the term refers to alleviating the symptoms associated with the specific disorder, disease, or condition. In some embodiments, the term refers to restoring function which was impaired or lost due to a specific disorder, disorder or condition.

The terms “enantiomers,” “stereoisomers,” and “optical isomers may be used interchangeably herein and refer to molecules which contain an asymmetric or chiral center and are mirror images of one another. Further, the terms “enantiomers,” “stereoisomers,” or “optical isomers” describe a molecule which, in a given configuration, cannot be superimposed on its mirror image.

The terms “optically pure” or “enantiomerically pure” may be understood herein to indicate that a composition contains at least 99.95% of a single optical isomer of a compound. The term “enantiomerically enriched” may be taken to indicate that a least 51% of a composition in a single optical isomer or enantiomer. The term “enantiomeric enrichment” refers herein to an increase in the amount of one enantiomer as compared to the other. A “racemic” mixture of 2 amino 4,5,6,7 tetrahydro 6 (propylamino)benzothiazole is a mixture of about equal amounts of (6R) and (6S) enantiomers of 2 amino 4,5,6,7 tetrahydro 6 (propylamino)benzothiazole.

Throughout this disclosure, the word “pramipexole” will refer to (6S) enantiomer of 2 amino 4,5,6,7 tetrahydro 6 (propylamino)benzothiazole unless otherwise specified.

The term “pharmaceutical composition” shall mean a composition including at least one active ingredient, whereby the composition is amenable to investigation for a specified, efficacious outcome in a human. Those of ordinary skill in the art will understand and appreciate the techniques appropriate for determining whether an active ingredient has a desired efficacious outcome based upon the needs of the artisan. A pharmaceutical composition may, for example, contain dexpramipexole or a pharmaceutically acceptable salt of dexpramipexole as the active ingredient. Alternatively, a pharmaceutical composition may contain dexpramipexole or a pharmaceutically acceptable salt of dexpramipexole as the active ingredient.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 119, describes pharmaceutically acceptable salts in detail. A pharmaceutical acceptable “salt” is any acid addition salt, preferably a pharmaceutically acceptable acid addition salt, including, but not limited to, halogenic acid salts such as hydrobromic, hydrochloric, hydrofloric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p toluenesufonic, acetic, malic, fumaric, succinic, citric, benzonic gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di hydrohalogic, di sulfuric, di phosphoric or di organic acid salt. In all cases, the acid addition salt is used as an achiral reagent which is not selected on the basis of any expected or known preference for the interaction with or precipitation of a specific optical isomer of the products of this disclosure.

The term “daily dose” refers to the amount of dexpramipexole, or pharmaceutically acceptable salt, per day that is administered to a patient. This amount can be administered in multiple unit doses or in a single unit doses, in a single time during the day or at multiple times during the day.

The term “hematologic responder human subject” refers to a human subject with moderate to severe asthma that has a greater than or equal to 50% reduction in absolute blood eosinophil level from baseline after administration of dexpramipexole, or a pharmaceutically acceptable salt thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

### Asthma and Effects of Dexpramipexole

As discussed herein, asthma is a heterogeneous disease, usually characterized by chronic airway inflammation. It is defined by the history of respiratory symptoms such as wheeze, shortness of breath, chest tightness, and cough that vary over time and in intensity, together with expiratory airflow limitation. Asthma is a condition in which the airways narrow due to airway smooth muscle constriction and mucosal swelling. The increase of mucus production in the airway lumen further contributes to obstructing airflow and worsening asthma symptoms. This can make breathing difficult and trigger coughing, wheezing and shortness of breath. For some people, asthma is a minor nuisance. For others, is a major problem that interferes with daily activities and that may lead to a life-threatening asthma attack or exacerbation. Current treatments cannot cure asthma, but its symptoms can be controlled in some patients.

Asthma signs and symptoms include: shortness of breath, chest tightness or pain, trouble sleeping caused by shortness of breath, coughing or wheezing (wheezing is a common sign of asthma in children), and exercise intolerance. Such signs and symptoms are typically worsened by viral upper respiratory infections, such as a cold or influenza. Asthmatics experience episodic exacerbations of their asthma, which consist of an increase in their asthma symptoms. Asthma exacerbations are a major cause of asthma morbidity and comprise a large fraction of asthma healthcare expense.

Control of asthma symptoms is typically assessed by a clinician based on a patient's FEV₁, peak expiratory flow, nighttime awakenings, short-acting bronchodilator rescue medication use, or other symptoms that would require an increase in inhaled corticosteroids (ICS) or oral corticosteroids (OCS) dose. Asthma severity is classified by the level of treatment required to control symptoms and exacerbations. The Global Initiative for Asthma (GINA) defines 5 steps of increasing intensity of treatment (GINA steps 1-5). Mild, moderate, and severe asthma correspond to GINA steps 1-2, 3, and 4-5, respectively. Many of the medications developed for asthma are taken by inhalation. Some medications developed for asthma require injection. Current treatment includes monoclonal antibodies, for example, benralizumab, which must be administered subcutaneously every 4 weeks, mepolizumab which must be administered subcutaneously every 4 weeks for 2 doses and 8 weeks thereafter; and reslizumab, which is administered intravenously every 4 weeks.

Accordingly, an oral drug that can reduce or eliminate the symptoms of severe asthma of the eosinophilic phenotype, such as decreasing the number of exacerbations requiring medical intervention, would be beneficial, particularly a drug that is administered orally and that can be used as a controller medication alone or in combination with the standard of care (such as an inhaled corticosteroid, often referred to as ICS), a long-acting beta-2 agonist (often referred to as a LABA), or a combination thereof. Described herein are methods of using dexpramipexole, or pharmaceutically acceptable salts thereof, in treating severe asthma of the eosinophilic phenotype.

Not wishing to be bound by theory, inflammation is an important component in the pathogenesis of asthma. IL-5 is the major cytokine responsible for the growth, proliferation, differentiation, recruitment, activation, and survival of eosinophils. Current asthma medications work in a variety of ways. By inhibiting IL-5 signaling, a reduction in the production and survival of eosinophils can be seen. Additional mechanisms include the binding to FcγRIII receptors on immune effectors cells, such as natural killer (NK) cells, leading to apoptosis of eosinophils through antibody-dependent cell-mediated cytotoxicity (ADCC), thus reducing the level of eosinophils. However, a reduction in the levels of eosinophils is not necessarily predictive of clinical success in all eosinophilic diseases. In a previous clinical trial directed to treating eosinophilic chronic rhinosinusitis with nasal polyps with 150 mg dexpramipexole twice daily, it was observed that despite a large and significant change in blood AEC, total polyp score (TPS) was unaffected. Chronic rhinosinusitis with nasal polyps is understood to be an eosinophilic disease and other eosinophil lowering drugs, such as mepolizumab, have shown efficacy. The KNS-760704-CS201 clinical trial was an open-label one arm multi-center 6 month trial of dexpramipexole 150 mg twice daily enrolling 16 subjects with eosinophilic chronic rhinosinusitis with nasal polyps. The co-primary endpoints were change in AEC and change in total polyp score (TPS) from baseline to Month 6, with additional clinical and histologic endpoints assessed. The trial met the first co-primary endpoint, demonstrating 94% lowering of the AEC after 6 months. In contrast to the large and significant change in blood AEC, TPS was unaffected at either the 3 or 6 month timepoints (Baseline 5.29; 3 months 5.50; 6 months 5.42 [mean values]). Other indices of chronic rhinosinusitis disease activity, including SNOT-22 (rhinosinusitis symptoms), sinus CT score, and Sniffin® Sticks olfaction test were also not improved. In the 12 subjects with a history of asthma, the ACQ6 and FEV₁ values were unchanged by treatment with dexpramipexole. See also Laidlaw, et al. Dexpramipexole Depletes Blood and Tissue Eosinophils in Nasal Polyps With No Change in Polyp Size, The Laryngoscope, 2019 February; 129 (2): E61-E66 (Epub 2018 Oct. 4).

Surprisingly, dexpramipexole further decreases the level of eosinophils not just in peripheral blood but also the tissue of the lungs and nasal passages, which results in superior efficacy in treating severe asthma of the eosinophilic phenotype, such as by decreasing blood absolute eosinophil count, decreasing the number of exacerbations, increasing FEV₁, increasing FVC, increasing peak expiratory flow, improving annualized CompEx event rate, and/or improving subject ACQ-7, ACQ-6 and AQLQ scores. The pathogenesis of severe asthma also includes the development of mucus plugs in the subject's airways, creating, in effect, a one-way valve, allowing air into the lungs but not efficiently out of the lungs, leading to air trapping and a decrease in forced vital capacity (FVC), and residual volume to total lung capacity ratio (RV/TLC). Dexpramipexole treats these mucus plugs, improves lung capacity, and decreases exacerbations requiring medical intervention. The nasal eosinophil peroxidase (EPX) measurement directly correlates with the level of sputum (tissue) eosinophils and can be utilized as a method to quickly and easily assess the level of tissue involvement and treatment success.

### Treatment of Inadequately Controlled Severe Asthma

Embodiments are directed to methods for treating inadequately controlled severe asthma of the eosinophilic phenotype in a human subject comprising orally administering to the human subject a daily dose of about 150 mg to about 300 mg of dexpramipexole, or a pharmaceutically acceptable salt thereof, wherein the human subject is already receiving at least two asthma medications, thereby treating the inadequately controlled severe asthma of the eosinophilic phenotype in the human subject. In some embodiments, the at least two asthma medications already being received by the inadequately controlled severe asthmatic human subject comprise, at a minimum: (1) medium dose inhaled corticosteroids on a regular basis for at least 12 months and on a stable dose for at least 3 months, and (2) an additional daily maintenance asthma medication selected from a long-acting β2 agonist, a leukotriene antagonist, theophylline, a long-action muscarinic antagonist, and cromolyn/nedocromil for at least 3 months.

In embodiments described herein, dexpramipexole or a pharmaceutically acceptable salt thereof, may be administered to the human subject at a daily dose of about 150 mg per day. In embodiments described herein, dexpramipexole or a pharmaceutically acceptable salt thereof, may be administered to the human subject at a daily dose of about 300 mg per day.

In embodiments described herein, dexpramipexole or a pharmaceutically acceptable salt thereof, may be administered to the human subject at a dose of about 75 mg twice per day, i.e., BID. In embodiments described herein, dexpramipexole or a pharmaceutically acceptable salt thereof, may be administered to the human subject at a dose of about 150 mg twice per day, i.e., BID.

In embodiments described herein, dexpramipexole is administered twice daily for up to 52 weeks. In embodiments described herein, dexpramipexole is administered twice daily for up to 44 weeks. In embodiments described herein, dexpramipexole is administered twice daily for up to 36 weeks. In embodiments described herein, dexpramipexole is administered twice daily for up to 28 weeks. In embodiments described herein, dexpramipexole is administered twice daily for up to 20 weeks. In embodiments described herein, dexpramipexole is administered twice daily for up to 12 weeks. In embodiments described herein, dexpramipexole is administered twice daily for up to 4 weeks. In embodiments described herein, dexpramipexole is administered twice daily until the inadequately controlled severe asthma of the eosinophilic phenotype is resolved. In embodiments described herein, dexpramipexole is administered twice daily for the lifetime of the subject.

In some embodiments, the inadequately controlled severe asthmatic human subject is defined as a subject with symptomatic severe asthma as defined by GINA steps 4 and 5. In some embodiments, the inadequately controlled severe asthmatic human subject may have a blood eosinophil count (AEC) of <0.30×10⁹ cells/L. In some embodiments, the inadequately controlled severe asthmatic human subject may have AEC of ≤0.30×10⁹ cells/L. For example, the inadequately controlled severe asthmatic human subject may have AEC of ≥0.15×10⁹ cells/L and <0.30×10⁹ cells/L. In another example, the inadequately controlled severe asthmatic human subject may have AEC of ≥0.15×10⁹ cells/L and ≤0.30×10⁹ cells/L. While previous studies have focused on treating subjects having AEC of ≥0.30×10⁹ cells/L, the methods described herein may have efficacy in patients having AEC of ≥0.15×10⁹ cells/L and <0.30×10⁹ cells/L. In some embodiments, improvement of one or more endpoints associated with treating the inadequately controlled severe asthma as further described herein may correlate with the measured AEC in the subject prior to administration. For example, a first subject having AEC of 0.15×10⁹ cells/L may exhibit a first magnitude of improvement in one or more endpoints, and a second subject having AEC of >0.15×10⁹ cells/L may exhibit a second magnitude of improvement in the one or more endpoints, wherein the second magnitude is greater than the first magnitude. However, it should also be understood that in some embodiments, the inadequately controlled severe asthmatic human subject is further defined as a subject with a blood eosinophil count (AEC) of ≥0.30×10⁹ cells/L.

The inadequately controlled severe asthmatic human subject may require daily treatment with, at a minimum: (1) medium dose inhaled corticosteroids on a regular basis for at least 12 months and on a stable dose for at least 3 months, and (2) an additional daily maintenance asthma medication selected from a long-acting β2 agonist, a leukotriene antagonist, theophylline, a long-action muscarinic antagonist, and cromolyn/nedocromil for at least 3 months. The inadequately controlled severe asthmatic human subject may have a pre-bronchodilator FEV₁≥40% and an FEV₁ of <80% of predicted. The inadequately controlled severe asthmatic human subject may have variable airflow obstruction evidenced by evidenced by, in the previous 12 months, at least one of: (1) ≥12% and ≥200 mL improvement in FEV₁ 15 to 30 minutes following inhalation of albuterol; (2) peak flow variation of ≥20% over a 2-week period and/or between clinic visits; (3) ACQ-6≥1.5; and (4) documented history of ≥2 asthma exacerbations requiring treatment with systemic corticosteroids (intramuscular, intravenous, or oral). In some embodiments, the inadequately controlled severe asthma of the eosinophilic phenotype is defined by the inclusion and exclusion criteria of Examples 3-4 (Tables E and F). In preferred embodiments, the important criteria include one or more selected from the group consisting of requiring a minimum daily medium dose ICS plus LABA treatment to maintain asthma control, severe asthma exacerbations of 2 or more times a year, ACQ score of ≥1.5, and AEC of ≥0.30×10⁹ cells/L.

Conventionally, subjects with severe asthma may be defined by a variety of characteristics and/or measurables, including but not limited to AEC. However, frequency of asthma exacerbations may also be a useful criterion for the effect of treatment of the severe asthma, i.e., whether inadequately controlled severe asthma is controlled by the treatment. Advantageously, the methods described herein further define inadequately controlled severe asthma in a subject based partly on a history of severe asthma exacerbations. For example, a subject having inadequately controlled severe asthma may have a history of severe asthma exacerbations two or more times per year and/or two or more severe asthma exacerbations in the past year. Furthermore, as further described herein, frequency of severe asthma exacerbations and/or quality of life may be assessed as meaningful endpoints to determine efficacy of treatment for the subject. As further discussed herein, the disclosed methods may result in an improvement in reduction in frequency of asthma exacerbations in a subject.

In embodiments described herein, the at least two asthma medications are an inhaled corticosteroids (ICS) and a long-acting beta agonist (LABA).

The inhaled corticosteroid (ICS) is selected from the group consisting of beclomethasone, fluticasone, ciclesonide, mometasone, budesonide, flunisolide, and combinations thereof. The inhaled corticosteroids may be medium-high dose inhaled corticosteroids or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be a medium dose or high dose.

In embodiments described herein, the inadequately controlled severe asthma of the eosinophilic phenotype in the subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) (up to 10 mg of prednisone per day or equivalent). In embodiments described herein, the inadequately controlled severe asthma of the eosinophilic phenotype in the subject may further be taking a long-acting muscarinic antagonist (LAMA) selected from the group consisting of umeclidinium, aclidinium, gycopyrronium, glycopyrrolate, tiotropium, and combinations thereof.

Surprisingly, beta-adrenergic agonist bronchodialator reversibility in subjects taking dexpramipexole is maintained. In other words, dexpramipexole provides a clinical benefit having a different mechanism of action compared with beta agonists, which are medications used by almost all asthmatic patients. Accordingly, the effect of dexpramipexole is additive when taken with other asthma medications.

In some embodiments, the subject is 12 years of age or older. In some embodiments, the subject is 12 years of age to about 17 years of age. In some embodiments, the subject is 12 years of age or older. In embodiments described herein, the subject is an adult or an adolescent. In embodiments described herein, the adolescent is from 12 up to 18 years of age. In embodiments described herein, the subject is 18 years of age or older. In embodiments described herein, the subject is between the ages of 18 to 75 years old. In embodiments described herein, the subject is between the ages of 12 to 75 years old. In embodiments described herein, the subject is 75 years of age or older.

Treatment of the inadequately controlled severe asthma may be assessed according to a primary endpoint. In some embodiments, the inadequately controlled severe asthma of the eosinophilic phenotype in the subject is treated, wherein the subject shows improvement in reduction in frequency of asthma exacerbations. In some embodiments, improvement in reduction in frequency of asthma exacerbations is based on a reduction in the number of exacerbations per year. In some embodiments, improvement in reduction in frequency of asthma exacerbations is determined by annualized rate of severe asthma exacerbations (AAER) over 52 weeks. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation. While previous studies have assessed treatment of the inadequately controlled severe asthma by improvement in other endpoints, the methods described herein advantageously assess the occurrence of severe asthma exacerbations, which may be a useful indicator of the overall efficacy of the methods for treating the inadequately controlled severe asthma in the subject, e.g., resulting in the severe asthma being controlled.

In some embodiments, improvement in reduction in frequency of asthma exacerbations is based on improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. An increase in asthma symptoms requiring oral or intravenous systemic corticosteroid use for at least 3 consecutive days is considered evidence of a severe asthma exacerbation for; for IM corticosteroids (longer acting), a single dose is sufficient. In some embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments described herein, exacerbations requiring the use of a systemic corticosteroid (e.g., OCS) as well as exacerbations resulting in hospitalization or an emergency room visit were each reduced.

Furthermore, treatment of the inadequately controlled severe asthma may be additionally or alternatively assessed according to one or more key secondary endpoints. In some embodiments, the inadequately controlled severe asthma of the eosinophilic phenotype in the subject is treated, wherein the inadequately controlled severe asthma of the eosinophilic phenotype in the subject shows improvement in at least one measurement selected from the group consisting of pre-bronchodilator forced expiratory volume in 1 second (FEV₁), score on Asthma Control Questionnaire (ACQ), score on Asthma Quality of Life Questionnaire (AQLQ), and combinations thereof.

Furthermore, treatment of the inadequately controlled severe asthma may be additionally or alternatively assessed according to one or more additional secondary endpoints. In some embodiments, the inadequately controlled severe asthma of the eosinophilic phenotype in the subject is treated, wherein the inadequately controlled severe asthma of the eosinophilic phenotype in the subject shows improvement in at least one measurement selected from the group consisting of AAER of severe exacerbations requiring an ED visit or hospitalization, forced vital capacity (FVC), post-bronchodilator FEV₁, peak expiratory flow (PEF), time to first exacerbation, total asthma symptom score, and EuroQol five-dimensional questionnaire (EQ-5D-5L), and combinations thereof.

In some embodiments, treatment of the inadequately controlled severe asthma may be additionally or alternatively assessed according to additional endpoints. In some embodiments, the inadequately controlled severe asthma of the eosinophilic phenotype in the subject is treated, wherein the subject shows improvement in a measurement selected from the group consisting of annualized CompEx event rate, morning peak expiratory flow (PEF), score of Asthma Quality of Life Questionnaire (AQLQ), score on St. George's Respiratory Questionnaire, level of nasal eosinophil peroxidase, level of pharyngeal eosinophil peroxidase, level of tissue eosinophils, amount of mucus plugs in the airways, level of blood basophils, level of blood eosinophils, level of absolute blood eosinophil, level of blood eosinophil progenitor population, fractional exhaled nitric oxide, and combinations thereof.

In some embodiments, treating the inadequately controlled severe asthma of the eosinophilic phenotype in the subject by oral administration of a daily dose of about 150 mg to about 300 mg of dexpramipexole results in a reduction of the level of eosinophils to less than 300 cells per microliter in the peripheral blood by a period of time selected from the group consisting of 44 weeks, 36 weeks, 28 weeks, 20 weeks, 12 weeks, 8 weeks, 6 weeks, 4 weeks, 3 weeks, and 2 weeks.

In some embodiments, treating the inadequately controlled severe asthma of the eosinophilic phenotype in the subject by oral administration of a daily dose of about 150 mg to about 300 mg of dexpramipexole results in a reduction of the level of eosinophils to less than 250 cells per microliter in the peripheral blood by a period of time selected from the group consisting of 44 weeks, 36 weeks, 28 weeks, 20 weeks, 12 weeks, 8 weeks, 6 weeks, 4 weeks, 3 weeks, and 2 weeks.

In some embodiments, treating the inadequately controlled severe asthma of the eosinophilic phenotype in the subject by oral administration of a daily dose of about 150 mg to about 300 mg of dexpramipexole results in a reduction of the level of eosinophils to less than 200 cells per microliter in the peripheral blood by a period of time selected from the group consisting of 44 weeks, 36 weeks, 28 weeks, 20 weeks, 12 weeks, 8 weeks, 6 weeks, 4 weeks, 3 weeks, and 2 weeks.

In some embodiments, treating the inadequately controlled severe asthma of the eosinophilic phenotype in the subject by oral administration of a daily dose of about 150 mg to about 300 mg of dexpramipexole results in a reduction of the level of eosinophils to less than 150 cells per microliter in the peripheral blood by a period of time selected from the group consisting of 44 weeks, 36 weeks, 28 weeks, 20 weeks, 12 weeks, 8 weeks, 6 weeks, 4 weeks, 3 weeks, and 2 weeks.

In some embodiments, treating the inadequately controlled severe asthma of the eosinophilic phenotype in the subject by oral administration of a daily dose of about 150 mg to about 300 mg of dexpramipexole results in a reduction of the level of eosinophils to less than 100 cells per microliter in the peripheral blood by a period of time selected from the group consisting of 44 weeks, 36 weeks, 28 weeks, 20 weeks, 12 weeks, 8 weeks, 6 weeks, 4 weeks, 3 weeks, and 2 weeks.

In some embodiments, treating the inadequately controlled severe asthma of the eosinophilic phenotype in the subject by administration of a daily dose of about 150 mg to about 300 mg of dexpramipexole results in a reduction of the level of absolute blood eosinophils by at least about 90%. In certain embodiments, treating the inadequately controlled severe asthma of the eosinophilic phenotype in the subject by administration of a daily dose of about 150 mg to about 300 mg of dexpramipexole results in a reduction of the level of absolute blood eosinophils by at least about 50%. In certain embodiments, treating the inadequately controlled severe asthma of the eosinophilic phenotype in the subject by administration of a daily dose of about 150 mg to about 300 mg of dexpramipexole results in a reduction of the level of absolute blood eosinophils by about 80%. In certain embodiments, treating the inadequately controlled severe asthma of the eosinophilic phenotype in the subject by administration of a daily dose of about 150 mg to about 300 mg of dexpramipexole results in a reduction of the level of absolute blood eosinophils by about 70%. In certain embodiments, treating the inadequately controlled severe asthma of the eosinophilic phenotype in the subject by administration of a daily dose of about 150 mg to about 300 mg of dexpramipexole results in a reduction of the level of absolute blood eosinophils by about 60%.

In some embodiments, treating the inadequately controlled severe asthma of the eosinophilic phenotype in the subject results in a reduction of the level of eosinophils in the tissue. In certain embodiments, treating the inadequately controlled severe asthma of the eosinophilic phenotype in the subject results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating the inadequately controlled severe asthma of the eosinophilic phenotype in the subject results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 150 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the inadequately controlled severe asthma of the eosinophilic phenotype in the subject is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the severe asthma of the eosinophilic phenotype in a human subject is treated and the nasal EPX levels are reduced to normal levels. In embodiments, the nasal and sputum EPX levels following administration of a daily dose of about 150 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas severe asthma of the eosinophilic phenotype human subject may be diagnosed as having nasal EPX levels greater than about 10 ng/mg of total protein. In some embodiments, is treated and the induced sputum EPX levels are reduced to the levels observed in a healthy, non-asthmatic control subject. Normal induced sputum EPX levels may be less than about 200 ng/ml/g recovered sputum, whereas severe asthma of the eosinophilic phenotype human subject is diagnosed as having induced sputum EPX levels greater than about 200 ng/ml/g recovered sputum. In certain embodiments, treating the inadequately controlled severe asthma of the eosinophilic phenotype in the subject by administration of a daily dose of about 300 mg of dexpramipexole results in a reduction of the level of nasal EPX by about 89%. In certain embodiments, treating the inadequately controlled severe asthma of the eosinophilic phenotype in the subject by administration of a daily dose of about 150 mg of dexpramipexole results in a reduction of the level of nasal EPX by about 83%. In certain embodiments, treating the inadequately controlled severe asthma of the eosinophilic phenotype in the subject by administration of a daily dose of about 150 mg of dexpramipexole results in a reduction of the level of nasal EPX by about 36%.

In some embodiments, is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including βc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating the inadequately controlled severe asthma of the eosinophilic phenotype in the subject comprising orally administering a daily dose of about 150 mg to about 300 mg of dexpramipexole, wherein the level of blood basophils is reduced.

In some embodiments, treating the inadequately controlled severe asthma of the eosinophilic phenotype in the subject by administration of a daily dose of about 150 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose are gradual and performed under the supervision of a physician. In embodiments described herein, reductions in oral corticosteroid dose and frequency occurs while maintaining asthma control.

In some embodiments, treating the inadequately controlled severe asthma of the eosinophilic phenotype in the subject by administration of a daily dose of about 150 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose are gradual and performed under the supervision of a physician. In embodiments described herein, reductions in inhaled corticosteroid dose and frequency occurs while maintaining asthma control.

In some embodiments, treating the inadequately controlled severe asthma of the eosinophilic phenotype in the subject by administration of a daily dose of about 150 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose are gradual and performed under the supervision of a physician. In embodiments described herein, reductions in long-acting beta agonist dose and frequency occurs while maintaining asthma control.

Embodiments described herein are directed to methods of treating the inadequately controlled severe asthma of the eosinophilic phenotype in the subject comprising orally administering a daily dose of about 150 mg to about 300 mg of dexpramipexole, wherein pulmonary function is improved. Pulmonary function is measured by performing a spirometry, which measures forced expiratory volume in 1s (FEV₁), the volume of air moved in the first second of exhalation, this is a well-accepted measure of asthma. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Embodiments described herein are directed to methods of treating the inadequately controlled severe asthma of the eosinophilic phenotype in the subject comprising orally administering a daily dose of about 150 mg to about 300 mg of dexpramipexole, wherein FEV₁ is improved. In certain embodiments, the inadequately controlled asthmatic human subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

Embodiments described herein are directed to methods of treating the inadequately controlled severe asthma of the eosinophilic phenotype in the subject comprising orally administering a daily dose of about 150 mg to about 300 mg of dexpramipexole, wherein FVC is improved. In certain embodiments, the inadequately controlled asthmatic human subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating the inadequately controlled severe asthma of the eosinophilic phenotype in the subject by administration of a daily dose of about 150 mg to about 300 mg of dexpramipexole results in an increase in morning peak expiratory flow (PEF). Peak flow zones are areas of measurement on a peak flow meter. The goal of the peak flow zones is to show early symptoms of inadequately controlled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating the inadequately controlled severe asthma of the eosinophilic phenotype in the subject by administration of a daily dose of about 150 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

Embodiments described herein are directed to methods of treating the inadequately controlled severe asthma of the eosinophilic phenotype in the subject comprising orally administering a daily dose of about 150 mg to about 300 mg of dexpramipexole, wherein the inadequately controlled severe asthma of the eosinophilic phenotype in the subject is controlled. The Asthma Control Questionnaire (ACQ), and/or the Asthma Control Test (ACT)™ can be used to assess whether asthma is being controlled. In certain embodiments, the inadequately controlled asthmatic human subject had a baseline score of about 1.9 to about 2.3 on the ACQ and about 4.5 to about 5.4 on the AQLQ.

The ACQ cutoff for controlled asthma is a score of about 0.75, and the ACQ cutoff for inadequately controlled asthma is a score of about 1.5. An ACQ score change of ≥0.5 is considered clinically significant. In embodiments described herein, the inadequately controlled asthmatic human subject has a change in ACQ score of at least 0.5 points. In embodiments described herein, the inadequately controlled asthmatic human subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the inadequately controlled asthmatic human subject has a change in ACT score of at least 0.5 points. In embodiments described herein, the inadequately controlled asthmatic human subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment.

Embodiments described herein are directed to methods of treating the inadequately controlled severe asthma of the eosinophilic phenotype in the subject comprising orally administering a daily dose of about 150 mg to about 300 mg of dexpramipexole, wherein the subject's quality of life is improved. The Asthma Quality of Life Questionnaire (AQLQ) can be used to assess an individual's quality of life, scores range from 1-7, with higher scores indicating better quality of life. In embodiments described herein, the inadequately controlled asthmatic human subject has a change in AQLQ score of at least 0.5 points. In embodiments described herein, the inadequately controlled asthmatic human subject has an improvement in score on Asthma Quality of Life Questionnaire (AQLQ) and the score is greater than 3.5.

In embodiments described herein, the inadequately controlled asthmatic human subject has an improvement in score on the St. George's Respiratory Questionnaire.

In some embodiments, treating the inadequately controlled severe asthma of the eosinophilic phenotype in the subject by administration of a daily dose of about 150 mg to about 300 mg of dexpramipexole results in improving a subject's overall morbidity. In some embodiments, treating the inadequately controlled severe asthma of the eosinophilic phenotype in the subject by administration of a daily dose of about 75 mg to about 300 mg of dexpramipexole results in improving a subject's overall mortality risk.

Surprisingly, treatment with dexpramipexole provides a remittive effect. Where treatment is discontinued following a predetermined period of treatment, the subject may continue to exhibit control of the severe asthma. In certain embodiments, the predetermined period of treatment is about 52 weeks. In additional embodiments, the predetermined period of treatment is about 48 weeks, about 44 weeks, about 40 weeks, about 36 weeks, about 32 weeks, about 28 weeks, about 24 weeks, about 20 weeks, about 16 weeks, about 12 weeks, about 8 weeks, or about 4 weeks. In certain embodiments, the severe asthma of the eosinophilic phenotype remains controlled after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In certain embodiments, the level of blood eosinophils remains reduced after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. Furthermore, where the subject restarts treatment following the discontinuation, the subject may exhibit a lack of tachyphylaxis, i.e., the subject responds to treatment in a substantially similar manner as prior to discontinuation (e.g., without a partial and/or progressive decrease in response).

In certain embodiments, the dexpramipexole is administered in combination with a bronchodilator selected from the group consisting of levalbuterol, ipratropium bromide, budesonide/formoterol, ipratropium, fluticasone/salmeterol, and combinations thereof. In certain embodiments, the dexpramipexole is administered in combination with an anti-inflammatory selected from the group consisting of zileuton, zafirlukast, and combinations thereof.

In certain embodiments, the dexpramipexole is administered instead of mepolizumab (NUCALA®). In certain embodiments, the dexpramipexole is administered instead of reslizumab (CINQAIR®). In certain embodiments, the dexpramipexole is administered instead of benralizumab (FASENRA®). In certain embodiments, the dexpramipexole is administered instead of dupilumab (DUPIXENT®). In certain embodiments, the dexpramipexole is administered instead of tezepelumab.

One of ordinary skill in the art will understand and appreciate the dosages and timing of the dosages to be administered to a human subject in need thereof. The doses and duration of treatment may vary, and may be based on assessment by one of ordinary skill in the art based on monitoring and measuring improvements in pulmonary and non-pulmonary tissues. This assessment may be made based on outward physical signs of improvement, such as decreased wheezing or less shortness of breath, or on internal physiological signs or markers. The doses may also depend on the condition or disease being treated, the degree of the condition or disease being treated and further on the age, weight, body mass index and body surface area of the subject.

In some embodiments, therapeutically effective amounts, or single unit doses of dexpramipexole may be administered multiple times per day, such as twice per day, i.e., BID. However, in additional embodiments, doses may be administered three times per day, four times per day, or five times per day.

Embodiments are also directed to a dosage regimen for orally administering dexpramipexole or a pharmaceutically acceptable salt thereof to treat the conditions disclosed herein. For example, in some embodiments, the methods described herein may comprise a dosage regimen that may include a plurality of daily doses having an equal amount of dexpramipexole or a pharmaceutically acceptable salt thereof as the initial dose in one or more unit doses. In other embodiments, the dosage regimen may include an initial dose of dexpramipexole or a pharmaceutically acceptable salt thereof in one or more unit doses, then a plurality of daily doses having a lower amount of dexpramipexole or a pharmaceutically acceptable salt thereof as the initial dose in one or more unit doses. The dosage regimen may administer an initial dose followed by one or more maintenance doses. The plurality of doses following the administering of an initial dose may be maintenance doses.

Such embodiments are not limited by the amount of the initial dose and daily doses. For example, in particular embodiments, the initial dose and each of the plurality of daily doses may be from about 150 mg to about 300 mg of dexpramipexole.

In some embodiments, two unit doses of about 75 mg are administered daily, wherein each unit dose may be substantially equal. In some embodiments, three unit doses totaling to about 150 mg are administered daily, wherein each unit dose may be substantially equal.

In some embodiments, two unit doses of about 150 mg are administered daily, wherein each unit dose may be substantially equal. In some embodiments, three unit doses totaling to about 300 mg are administered daily, wherein each unit dose may be substantially equal.

Following the initial dose for a period of time, the maintenance dose may include administering less than the initial daily dose of 150 mg to about 300 mg administered twice per day as a 37.5 mg, 75 mg, or 150 mg tablet, respectively, or administering the daily dose once per day. In preferred embodiments, the maintenance dose is 75 mg once per day, 150 mg, once per day, or 300 mg once per day.

In further embodiments, the method may include an initial dosing regimen and a maintenance dosing regimen. In certain embodiments, the initial dosing regimen may include administering a higher dose of dexpramipexole or a pharmaceutically acceptable salt thereof than the maintenance dosing regimen as either a single administration or by administering an increased dosage for a limited period of time prior to beginning a maintenance dosing regimen of dexpramipexole or a pharmaceutically acceptable salt thereof. In some embodiments, subjects undergoing a maintenance regimen may be administered one or more higher-dosage treatments at one or more times during the maintenance dosage regimen.

In some embodiments, the initial dosing regimen and the maintenance dosing regimen may include administering dexpramipexole or a pharmaceutically acceptable salt thereof once per day, or twice per day. In such embodiments, the dosage regimen may continue administering an initial dose for 1, 2, 3, 4, 5, 6 or 7 days, up to 4 weeks, up to 8 weeks or up to 12 weeks. In some embodiments, the dosage regimen for administering an initial dose and/or a maintenance dose may continue for an extended period of time. Various embodiments are directed to a dosing regimen for dexpramipexole or a pharmaceutically acceptable salt thereof in which maintenance doses are maintained for an extended period of time without titration or otherwise changing the dosing regimen. In such embodiments, the extended period of time may be about 12 weeks or longer, about 6 months or longer, about 1 year or longer, 2, 3, 4, 5, or 10 years or longer, and in certain embodiments, an indefinite period of time.

In some embodiments, treatment with a daily dose of about 150 mg to about 300 mg of dexpramipexole is without the adverse side effects associated with dopamine agonism.

Oral pharmaceutical compositions containing dexpramipexole or a pharmaceutically acceptable salt thereof in a solid dosage may include, but are not limited to, softgels, tablets, capsules, cachets, pellets, pills, powders and granules; other oral dosage forms include, but are not limited to, solutions, suspensions, emulsions, and dry powder.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

Additional information regarding treatment of asthma of the eosinophilic phenotype described herein can be found in International Patent Application No. PCT/US2021/044719 entitled “Use of Dexpramipexole for the Treatment of Moderate to Severe Asthma,” filed Aug. 5, 2021, which is hereby incorporated herein by reference in its entirety.

### Treatment of Inadequately Controlled Moderate to Severe Eosinophilic Asthma

Embodiments are directed to methods for treating inadequately controlled moderate to severe asthma of the eosinophilic phenotype in a human subject comprising orally administering to the human subject a daily dose of about 150 mg to about 300 mg of dexpramipexole, or a pharmaceutically acceptable salt thereof, wherein the human subject is already receiving at least two asthma medications, thereby treating the inadequately controlled moderate to severe asthma of the eosinophilic phenotype in the human subject. In some embodiments, the at least two asthma medications already being received by the inadequately controlled moderate to severe asthmatic human subject comprise, at a minimum: (1) medium dose inhaled corticosteroids on a regular basis for at least 12 months and on a stable dose for at least 3 months, and (2) an additional daily maintenance asthma medication selected from a long-acting β2 agonist, a leukotriene antagonist, theophylline, a long-action muscarinic antagonist, and cromolyn/nedocromil for at least 3 months.

In embodiments described herein, dexpramipexole or a pharmaceutically acceptable salt thereof, may be administered to the human subject at a daily dose of about 150 mg per day. In embodiments described herein, dexpramipexole or a pharmaceutically acceptable salt thereof, may be administered to the human subject at a daily dose of about 300 mg per day.

In embodiments described herein, dexpramipexole or a pharmaceutically acceptable salt thereof, may be administered to the human subject at a dose of about 75 mg twice per day, i.e., BID. In embodiments described herein, dexpramipexole or a pharmaceutically acceptable salt thereof, may be administered to the human subject at a dose of about 150 mg twice per day, i.e., BID.

In embodiments described herein, dexpramipexole is administered twice daily for up to 24 weeks. In embodiments described herein, dexpramipexole is administered twice daily for up to 20 weeks. In embodiments described herein, dexpramipexole is administered twice daily for up to 16 weeks. In embodiments described herein, dexpramipexole is administered twice daily for up to 12 weeks. In embodiments described herein, dexpramipexole is administered twice daily for up to 8 weeks. In embodiments described herein, dexpramipexole is administered twice daily for up to 4 weeks. In embodiments described herein, dexpramipexole is administered twice daily until the inadequately controlled moderate to severe asthma of the eosinophilic phenotype is resolved. In embodiments described herein, dexpramipexole is administered twice daily for the lifetime of the subject.

In some embodiments, the inadequately controlled moderate to severe asthmatic human subject is defined as a subject with symptomatic moderate to severe asthma as defined by GINA steps 3, 4 and 5. In some embodiments, the inadequately controlled moderate to severe asthmatic human subject is further defined as a subject with a blood eosinophil count (AEC) of ≥0.25×10⁹ cells/L. In some embodiments, the inadequately controlled moderate to severe asthmatic human subject is further defined as a subject with a blood eosinophil count (AEC) of ≥0.30×10⁹ cells/L.

The inadequately controlled moderate to severe asthmatic human subject may require daily treatment with, at a minimum: (1) low dose inhaled corticosteroids on a regular basis for at least 12 weeks and on a stable dose for at least 1 week, and (2) an additional daily maintenance asthma medication selected from a long-acting β2 agonist, a leukotriene antagonist, theophylline, a long-action muscarinic antagonist, and cromolyn/nedocromil on a stable dose for at least 4 weeks. The inadequately controlled moderate to severe asthmatic human subject may have a pre-bronchodilator FEV₁≥40% and an FEV₁ of <80% of predicted. The inadequately controlled moderate to severe asthmatic human subject may have variable airflow obstruction evidenced by evidenced by, in the previous 12 months, at least one of: (1) ≥12% and ≥200 mL improvement in FEV₁ 15 to 30 minutes following inhalation of albuterol (≥160 mL for ages 12 to 17); (2) ACQ-6≥1.5; and (3) blood eosinophil count (AEC) of at least ≥0.25×10⁹ cells/L, preferably ≥0.30×10⁹ cells/L. In some embodiments, the inadequately controlled moderate to severe asthma of the eosinophilic phenotype is defined by the inclusion and exclusion criteria of Example 5 (Table G). In preferred embodiments, the important criteria include one or more selected from the group consisting of requiring a minimum daily medium dose ICS plus LABA treatment to maintain asthma control, ACQ score of ≥1.5, and AEC of ≥0.30×10⁹ cells/L.

In embodiments described herein, the at least two asthma medications are an inhaled corticosteroid (ICS) and a long-acting beta agonist (LABA).

The inhaled corticosteroids (ICS) is selected from the group consisting of beclomethasone, fluticasone, ciclesonide, mometasone, budesonide, flunisolide, and combinations thereof. The inhaled corticosteroids may be medium-high dose inhaled corticosteroids or high-dose ICS. The long-acting beta agonist (LABA) is selected from the group consisting of albuterol sulfate, formoterol fumarate, salmeterol, salmeterol xinafoate, arformoterol tartrate, olodaterol, vilanterol, indacaterol, and combinations thereof. The amount of the inhaled corticosteroid being taken by the subject may be a medium dose or high dose.

In embodiments described herein, the inadequately controlled moderate to severe asthma of the eosinophilic phenotype in the subject may further be taking an oral corticosteroid (OCS) selected from the group consisting of cortisone acetate, dexamethasone, hydrocortisone, methylprednisolone, prednisolone, prednisone, and combinations thereof. Maintenance oral corticosteroids (OCS) (up to 10 mg of prednisone per day or equivalent). In embodiments described herein, the inadequately controlled moderate to severe asthma of the eosinophilic phenotype in the subject may further be taking a long-acting muscarinic antagonist (LAMA) selected from the group consisting of umeclidinium, aclidinium, gycopyrronium, glycopyrrolate, tiotropium, and combinations thereof.

Surprisingly, beta-adrenergic agonist bronchodialator reversibility in subjects taking dexpramipexole is maintained. In other words, dexpramipexole provides a clinical benefit having a different mechanism of action compared with beta agonists, which are medications used by almost all asthmatic patients. Accordingly, the effect of dexpramipexole is additive when taken with other asthma medications.

In some embodiments, the subject is 12 years of age or older. In some embodiments, the subject is 12 years of age to about 17 years of age. In some embodiments, the subject is 12 years of age or older. In embodiments described herein, the subject is an adult or an adolescent. In embodiments described herein, the adolescent is from 12 up to 18 years of age. In embodiments described herein, the subject is 18 years of age or older. In embodiments described herein, the subject is between the ages of 18 to 75 years old. In embodiments described herein, the subject is between the ages of 12 to 75 years old. In embodiments described herein, the subject is 75 years of age or older.

Treatment of the inadequately controlled moderate to severe asthma may be assessed according to a primary endpoint. In some embodiments, the inadequately controlled moderate to severe asthma of the eosinophilic phenotype in the subject is treated, wherein the subject shows improvement in pulmonary function. In some embodiments, the inadequately controlled moderate to severe asthma of the eosinophilic phenotype in the subject is treated, wherein the inadequately controlled moderate to severe asthma of the eosinophilic phenotype in the subject shows improvement in pre-bronchodilator forced expiratory volume in 1 second (FEV₁). Pulmonary function is measured by performing a spirometry, which measures forced expiratory volume in 1 second (FEV₁), the volume of air moved in the first second of exhalation. However, additional measures may be utilized as well. Forced vital capacity (FVC) is the total volume of air exhaled during the entire forced expiratory volume (FEV) test. The FEV₁/FVC ratio is a calculated measure that may provide a more specific measure of bronchoconstriction, independent of processes such as air trapping. Pulmonary function testing can be tested pre-bronchodilator (after waiting a sufficient time so that all bronchodilator medications have cleared) or post-bronchodilator, typically 15-30 minutes after inhaling a short-acting bronchodilator, such as albuterol. Bronchodilator reversibility (BDR) is calculated and assessed by analyzing FEV₁ pre- and post-bronchodilator, and calculating the change both as absolute (ml) and percentage change.

Furthermore, treatment of the inadequately controlled moderate to severe asthma may be additionally or alternatively assessed according to one or more key secondary endpoints. In some embodiments, the inadequately controlled moderate to severe asthma of the eosinophilic phenotype in the subject is treated, wherein the inadequately controlled moderate to severe asthma of the eosinophilic phenotype in the subject shows improvement in at least one measurement selected from the group consisting of score on Asthma Control Questionnaire (ACQ), score on Asthma Quality of Life Questionnaire (AQLQ), and combinations thereof.

Furthermore, treatment of the inadequately controlled moderate to severe asthma may be additionally or alternatively assessed according to one or more additional secondary endpoints. In some embodiments, the inadequately controlled moderate to severe asthma of the eosinophilic phenotype in the subject is treated, wherein the inadequately controlled moderate to severe asthma of the eosinophilic phenotype in the subject shows improvement in at least one measurement selected from the group consisting of AEC, forced vital capacity (FVC), post-bronchodilator FEV₁, pre-bronchodilator FEV₁, and EuroQol five-dimensional questionnaire (EQ-5D-5L), and combinations thereof.

Furthermore, treatment of the inadequately controlled moderate to severe asthma may be additionally or alternatively assessed according to one or more exploratory endpoints. In some embodiments, the inadequately controlled moderate to severe asthma of the eosinophilic phenotype in the subject is treated, wherein the inadequately controlled moderate to severe asthma of the eosinophilic phenotype in the subject shows improvement in at least one measurement selected from the group consisting of annualized rate of severe asthma exacerbations (AAER) over 24 weeks, pharmacokinetic parameters including but not limited to maximum plasma concentration (Cₘₐₓ), time of maximum plasma concentration (Tₘₐₓ), area under the curve to the end of dosing (AUC₀ₜ), and trough concentration, and combinations thereof.

In some embodiments, treatment of the inadequately controlled severe asthma may be additionally or alternatively assessed according to additional endpoints. In some embodiments, the inadequately controlled severe asthma of the eosinophilic phenotype in the subject is treated, wherein the subject shows improvement in a measurement selected from the group consisting of reduction in frequency of severe asthma exacerbations, annualized CompEx event rate, morning peak expiratory flow (PEF), score on St. George's Respiratory Questionnaire, level of nasal eosinophil peroxidase, level of pharyngeal eosinophil peroxidase, level of tissue eosinophils, amount of mucus plugs in the airways, level of blood basophils, level of blood eosinophils, level of absolute blood eosinophil, level of blood eosinophil progenitor population, fractional exhaled nitric oxide, and combinations thereof.

In some embodiments, treating the inadequately controlled moderate to severe asthma of the eosinophilic phenotype in the subject by oral administration of a daily dose of about 150 mg to about 300 mg of dexpramipexole results in a reduction of the level of eosinophils to less than 300 cells per microliter in the peripheral blood by a period of time selected from the group consisting of 24 weeks, 20 week, 16 weeks, 12 weeks, 8 weeks, 6 weeks, 4 weeks, 3 weeks, and 2 weeks.

In some embodiments, treating the inadequately controlled moderate to severe asthma of the eosinophilic phenotype in the subject by oral administration of a daily dose of about 150 mg to about 300 mg of dexpramipexole results in a reduction of the level of eosinophils to less than 250 cells per microliter in the peripheral blood by a period of time selected from the group consisting of 24 weeks, 20 week, 16 weeks, 12 weeks, 8 weeks, 6 weeks, 4 weeks, 3 weeks, and 2 weeks.

In some embodiments, treating the inadequately controlled moderate to severe asthma of the eosinophilic phenotype in the subject by oral administration of a daily dose of about 150 mg to about 300 mg of dexpramipexole results in a reduction of the level of eosinophils to less than 200 cells per microliter in the peripheral blood by a period of time selected from the group consisting of 24 weeks, 20 week, 16 weeks, 12 weeks, 8 weeks, 6 weeks, 4 weeks, 3 weeks, and 2 weeks.

In some embodiments, treating the inadequately controlled moderate to severe asthma of the eosinophilic phenotype in the subject by oral administration of a daily dose of about 150 mg to about 300 mg of dexpramipexole results in a reduction of the level of eosinophils to less than 150 cells per microliter in the peripheral blood by a period of time selected from the group consisting of 24 weeks, 20 week, 16 weeks, 12 weeks, 8 weeks, 6 weeks, 4 weeks, 3 weeks, and 2 weeks.

In some embodiments, treating the inadequately controlled moderate to severe asthma of the eosinophilic phenotype in the subject by oral administration of a daily dose of about 150 mg to about 300 mg of dexpramipexole results in a reduction of the level of eosinophils to less than 100 cells per microliter in the peripheral blood by a period of time selected from the group consisting of 24 weeks, 20 week, 16 weeks, 12 weeks, 8 weeks, 6 weeks, 4 weeks, 3 weeks, and 2 weeks.

In some embodiments, treating the inadequately controlled moderate to severe asthma of the eosinophilic phenotype in the subject by administration of a daily dose of about 150 mg to about 300 mg of dexpramipexole results in a reduction of the level of absolute blood eosinophils by at least about 90%. In certain embodiments, treating the inadequately controlled moderate to severe asthma of the eosinophilic phenotype in the subject by administration of a daily dose of about 150 mg to about 300 mg of dexpramipexole results in a reduction of the level of absolute blood eosinophils by at least about 50%. In certain embodiments, treating the inadequately controlled moderate to severe asthma of the eosinophilic phenotype in the subject by administration of a daily dose of about 150 mg to about 300 mg of dexpramipexole results in a reduction of the level of absolute blood eosinophils by about 80%. In certain embodiments, treating the inadequately controlled moderate to severe asthma of the eosinophilic phenotype in the subject by administration of a daily dose of about 150 mg to about 300 mg of dexpramipexole results in a reduction of the level of absolute blood eosinophils by about 70%. In certain embodiments, treating the inadequately controlled moderate to severe asthma of the eosinophilic phenotype in the subject by administration of a daily dose of about 150 mg to about 300 mg of dexpramipexole results in a reduction of the level of absolute blood eosinophils by about 60%.

In some embodiments, treating the inadequately controlled moderate to severe asthma of the eosinophilic phenotype in the subject results in a reduction of the level of eosinophils in the tissue. In certain embodiments, treating the inadequately controlled moderate to severe asthma of the eosinophilic phenotype in the subject results in a reduction of the level of eosinophils in tissues including, but not limited to, lung tissue, nasal tissue, bone marrow, and combinations thereof. In certain embodiments, the level of eosinophils in the tissues are reduced by at least 40%, at least 45%, at least 50%, at least 55%, at least 60%, at least 65%, at least 70%, at least 75%, at least 80%, at least 85%, at least 90%, at least 95%, or 100%. In certain embodiments, the levels are reduced to normal. In certain embodiments, the levels are reduced to zero within the level of detection.

In some embodiments, treating the inadequately controlled moderate to severe asthma of the eosinophilic phenotype in the subject results in a reduction in the amount of mucus plugs in the airways. Without wishing to be bound by theory, mucus plugs contribute to chronic airflow obstruction in severe asthma, and eosinophils release eosinophil peroxidase (EPX), which generates reactive oxygen species that oxidize cysteine thiol groups, this leads to cross-linking of the mucin proteins, making the mucus stiffer and promoting mucus plug formation in the bronchi (airways). In embodiments described herein, orally administering a daily dose of about 150 mg to about 300 mg of dexpramipexole, or a pharmaceutically acceptable salt thereof, decreases the level of tissue eosinophils, thus reducing airway eosinophil peroxidase, which in turn, reduces the amount of mucus plugs in the airways, all of which leads to improved lung function and greater exercise tolerance.

In some embodiments, the inadequately controlled moderate to severe asthma of the eosinophilic phenotype in the subject is treated wherein the concentration of induced sputum eosinophil peroxidase (EPX), nasal eosinophil peroxidase (EPX), or pharyngeal eosinophil peroxidase (EPX) is reduced. Eosinophil peroxidase (EPX) is a granule protein uniquely secreted by eosinophils which can be detected and quantified using an enzyme-linked immunosorbent assay (ELISA). EPX is measured from mucosal swabs or sputum samples. In some embodiments, the moderate to severe asthma of the eosinophilic phenotype in a human subject is treated and the nasal EPX levels are reduced to normal levels. In embodiments, the nasal and sputum EPX levels following administration of a daily dose of about 150 mg to about 300 mg of dexpramipexole are reduced to level of the nasal and sputum EPX levels in a healthy, non-asthmatic control subject. In embodiments, normal nasal EPX levels are less than about 10 ng/mg of total protein, whereas moderate to severe asthma of the eosinophilic phenotype human subject may be diagnosed as having nasal EPX levels greater than about 10 ng/mg of total protein. In some embodiments, is treated and the induced sputum EPX levels are reduced to the levels observed in a healthy, non-asthmatic control subject. Normal induced sputum EPX levels may be less than about 200 ng/mL/g recovered sputum, whereas moderate to severe asthma of the eosinophilic phenotype human subject is diagnosed as having induced sputum EPX levels greater than about 200 ng/mL/g recovered sputum. In certain embodiments, treating the inadequately controlled moderate to severe asthma of the eosinophilic phenotype in the subject by administration of a daily dose of about 300 mg of dexpramipexole results in a reduction of the level of nasal EPX by about 89%. In certain embodiments, treating the inadequately controlled moderate to severe asthma of the eosinophilic phenotype in the subject by administration of a daily dose of about 150 mg of dexpramipexole results in a reduction of the level of nasal EPX by about 83%. In certain embodiments, treating the inadequately controlled moderate to severe asthma of the eosinophilic phenotype in the subject by administration of a daily dose of about 150 mg of dexpramipexole results in a reduction of the level of nasal EPX by about 36%.

In some embodiments, is treated wherein the level of urine eosinophil granule proteins are reduced. In certain embodiments, the eosinophil granule proteins are selected from the group consisting of MBP-1 (Eosinophil Major Basic Protein-1), EDN (Eosinophil-derived Neurotoxin, RNase-2), ECP (Eosinophil Cationic Protein, RNase-3), EPX (Eosinophil Peroxidase), Charcot-Leyden Crystal protein (Gal-10/CLC) and combinations thereof.

In some embodiments, the reduction in blood eosinophils correlates with a reduction in tissue eosinophil biomarkers. In some embodiments, the tissue eosinophil biomarkers are selected from the group consisting of type 2 inflammation associated mediators, including IL-5, IL-13, IL-33, ST2 (IL1RL1), CCL2, CCL3, CCL4, CCL11, CCL17, CCR3, and combinations thereof. Eosinophil development is supported by a variety of cytokines, including βc-related cytokines, such as GM-CSF, IL-3, and IL-5, which can also be used as biomarkers to assess the reduction in blood eosinophils. In some embodiments, the reduction in blood eosinophils correlates with a reduction in blood eosinophil progenitor populations. Eosinophils are derived from hematopoietic stem cells (HSCs), and both eosinophils and basophils are derived from myeloid progenitor cells.

In some embodiments, the methods of treating the inadequately controlled moderate to severe asthma of the eosinophilic phenotype in the subject comprising orally administering a daily dose of about 150 mg to about 300 mg of dexpramipexole, wherein the level of blood basophils is reduced.

In some embodiments, treating the inadequately controlled moderate to severe asthma of the eosinophilic phenotype in the subject by administration of a daily dose of about 150 mg to about 300 mg of dexpramipexole results in a reduction in oral corticosteroid dose. In some embodiments, the oral corticosteroid dose is reduced by about 90% to about 100%, about 75% to about 90%, about 50% to about 75%, or about 10% to about 50%. In some embodiments, the oral corticosteroid dose is reduced to zero. In some embodiments, the frequency of the use of oral corticosteroid is reduced from baseline, such as a 90-100% decrease, a 75-90% decrease, a 50-75% decrease, or at least a 50% reduction. In embodiments described herein, use of oral corticosteroids is reduced to every other day. In embodiments described herein, reductions in oral corticosteroid dose are gradual and performed under the supervision of a physician. In embodiments described herein, reductions in oral corticosteroid dose and frequency occurs while maintaining asthma control.

In some embodiments, treating the inadequately controlled moderate to severe asthma of the eosinophilic phenotype in the subject by administration of a daily dose of about 150 mg to about 300 mg of dexpramipexole results in a reduction in inhaled corticosteroid dose. In some embodiments, the inhaled corticosteroid dose is reduced by about 90% to about 100%, about 75% to about 90%, about 50% to about 75%, or about 10% to about 50%. In some embodiments, the inhaled corticosteroid dose is reduced to zero. In some embodiments, the frequency of the use of inhaled corticosteroid is reduced from baseline, such as a 90-100% decrease, a 75-90% decrease, a 50-75% decrease, or at least a 50% reduction. In embodiments described herein, the use of inhaled corticosteroids is reduced to less than 3 times per week. In embodiments described herein, reductions in inhaled corticosteroid dose are gradual and performed under the supervision of a physician. In embodiments described herein, reductions in inhaled corticosteroid dose and frequency occurs while maintaining asthma control.

In some embodiments, treating the inadequately controlled moderate to severe asthma of the eosinophilic phenotype in the subject by administration of a daily dose of about 150 mg to about 300 mg of dexpramipexole results in a reduction in long-acting beta agonist dose. In some embodiments, the long-acting beta agonist dose is reduced by about 90% to about 100%, about 75% to about 90%, about 50% to about 75%, or about 10% to about 50%. In some embodiments, the long-acting beta agonist dose is reduced to zero. In some embodiments, the frequency of the use of long-acting beta agonist is reduced from baseline, such as a 90-100% decrease, a 75-90% decrease, a 50-75% decrease, or at least a 50% reduction. In embodiments described herein, the use of long-acting beta agonist is reduced to less than 1 time per day. In embodiments described herein, reductions in long-acting beta agonist dose are gradual and performed under the supervision of a physician. In embodiments described herein, reductions in long-acting beta agonist dose and frequency occurs while maintaining asthma control.

Embodiments described herein are directed to methods of treating the inadequately controlled moderate to severe asthma of the eosinophilic phenotype in the subject comprising orally administering a daily dose of about 150 mg to about 300 mg of dexpramipexole, wherein FEV₁ is improved. In certain embodiments, the inadequately controlled asthmatic human subject had a baseline FEV₁ of about 1.9 L to about 2.2 L at baseline, which is about a 60% reduction from normal/predicted. In some embodiments, the FEV₁ is increased by about 5% to about 20%. More preferably, the FEV₁ is increased by about 10% to about 20%. In some embodiments, the volume of FEV₁ is increased by about 150 ml to about 300 ml. More preferably, the FEV₁ is increased by about 200 ml to about 300 ml.

Embodiments described herein are directed to methods of treating the inadequately controlled moderate to severe asthma of the eosinophilic phenotype in the subject comprising orally administering a daily dose of about 150 mg to about 300 mg of dexpramipexole, wherein FVC is improved. In certain embodiments, the inadequately controlled asthmatic human subject had a baseline FVC of about 3.0 L to about 3.3 L, which is about a 74%-80% reduction from normal/predicted. In some embodiments, the FVC is increased by about 2% to about 12%. More preferably, the FVC is increased by about 8% to about 12%. In some embodiments, the volume of FVC is increased by about 20 ml to about 450 ml. More preferably, the volume of FVC is increased by about 200 ml to about 450 ml.

In some embodiments, treating the inadequately controlled moderate to severe asthma of the eosinophilic phenotype in the subject by administration of a daily dose of about 150 mg to about 300 mg of dexpramipexole results in an increase in morning peak expiratory flow (PEF). Peak flow zones are areas of measurement on a peak flow meter. The goal of the peak flow zones is to show early symptoms of inadequately controlled asthma. The green zone is 80% to 100% of the subject's highest peak flow reading. The yellow zone is 50% to 80% of the subject's highest peak flow reading. Measurements in this zone are a sign that large airways are starting to narrow and subject may start to have mild symptoms, such as coughing, feeling tired, feeling short of breath, or chest tightening. The red zone is less than 50% of the subject's highest peak flow reading. Readings in this zone mean severe narrowing of large airways, which is a medical emergency. In some embodiments, treating the inadequately controlled moderate to severe asthma of the eosinophilic phenotype in the subject by administration of a daily dose of about 150 mg to about 300 mg of dexpramipexole results in the subject's ability to remain in the green zone when measuring morning peak expiratory flow.

Airway inflammation can also be assessed using the FeNO test, fractional concentration of exhaled nitric oxide, wherein the level of nitric oxide is measured in parts per billion (ppb) in the air you slowly exhale. Higher than normal levels of exhaled nitric oxide generally means the airways are inflamed, a sign of asthma. Levels less than about 20 ppb in children and less than about 25 ppb in adults are considered normal. Greater than about 35 ppb in children and 50 ppb in adults are considered to be abnormal.

In some embodiments, the inadequately controlled moderate to severe asthma of the eosinophilic phenotype in the subject is treated, wherein the subject shows improvement in reduction in frequency of asthma exacerbations. In some embodiments, improvement in reduction in frequency of asthma exacerbations is based on a reduction in the number of exacerbations per year. In some embodiments, improvement in reduction in frequency of asthma exacerbations is determined by annualized rate of severe asthma exacerbations (AAER) over 52 weeks. In some embodiments, improvement in exacerbations measured by either fewer exacerbations per unit of time or an extension in the time between the next subsequent exacerbation. In some embodiments, the exacerbation rate is reduced to less than 2 per year, less than 1 per year, or is 0 per year. In some embodiments, the time to first severe asthma exacerbation was extended. In some embodiments, the time to first severe asthma exacerbation did not occur within one year of the previous exacerbation.

In some embodiments, improvement in reduction in frequency of asthma exacerbations is based on improvement in the subject's annualized CompEx event rate. Annualized CompEx event rate is a measurement of an extended definition of asthma exacerbations, combining diary-based events with traditionally defined severe exacerbations. Diary events are based on objective measures of deterioration of peak expiratory flow, reliever use, and asthma symptoms assessed morning and evening and night-time awakenings. Deterioration is defined as either reaching a predefined change from baseline (threshold), for at least 2 consecutive days, or deterioration in all variables over at least a 5-day period plus at least one variable reaching a threshold criterion for at least 2 consecutive days. Deterioration of at least two concurrent criteria is needed to fulfill the criteria for a diary event.

A severe asthma exacerbation is defined as worsening of asthma requiring the use of systemic corticosteroids and/or hospitalization and/or medical intervention and/or emergency department (ED) visit. In certain embodiments, the medical intervention was corroborated with at least 1 of the following: 1) a decrease in forced expiratory volume in 1 second (FEV₁) by 20% or more from baseline, 2) a decrease in peak expiratory flow rate (PEFR) by 30% or more from baseline on 2 consecutive days, or 3) worsening of symptoms or other clinical signs per physician evaluation of the event. An increase in asthma symptoms requiring oral or intravenous systemic corticosteroid use for at least 3 consecutive days is considered evidence of a severe asthma exacerbation for; for IM corticosteroids (longer acting), a single dose is sufficient. In some embodiments, an exacerbation is defined as the worsening of asthma symptoms and lung function requiring use of oral/systemic corticosteroids for at least 3 days. In embodiments, for subjects on maintenance oral corticosteroids, an exacerbation requiring oral/systemic corticosteroids is defined as the use of oral/systemic corticosteroids that is at least double the existing dose for at least 3 days. In embodiments described herein, exacerbations requiring the use of a systemic corticosteroid (e.g., OCS) as well as exacerbations resulting in hospitalization or an emergency room visit were each reduced.

Embodiments described herein are directed to methods of treating the inadequately controlled moderate to severe asthma of the eosinophilic phenotype in the subject comprising orally administering a daily dose of about 150 mg to about 300 mg of dexpramipexole, wherein the inadequately controlled moderate to severe asthma of the eosinophilic phenotype in the subject is controlled. The Asthma Control Questionnaire (ACQ), and/or the Asthma Control Test (ACT)™ can be used to assess whether asthma is being controlled. In certain embodiments, the inadequately controlled asthmatic human subject had a baseline score of about 1.9 to about 2.3 on the ACQ and about 4.5 to about 5.4 on the AQLQ.

The ACQ cutoff for controlled asthma is a score of about 0.75, and the ACQ cutoff for inadequately controlled asthma is a score of about 1.5. An ACQ score change of ≥0.5 is considered clinically significant. In embodiments described herein, the inadequately controlled asthmatic human subject has a change in ACQ score of at least 0.5 points. In embodiments described herein, the inadequately controlled asthmatic human subject has an improvement in score on ACQ and the score is less than or equal to 0.75.

Using the Asthma Control Test (ACT)™, a score of less than 19 indicates the individual's asthma may not be well controlled and a score of less than 15 indicates the individual's asthma may be very poorly controlled. In embodiments described herein, the inadequately controlled asthmatic human subject has a change in ACT score of at least 0.5 points. In embodiments described herein, the inadequately controlled asthmatic human subject has an improvement in score on Asthma Control Test (ACT)™ and the score is greater than 19.

The AQLQ is a 32-item asthma specific questionnaire designed to measure functional impairments that are most important to patients with asthma. The 32 questions in the AQLQ are divided into four domains; activity limitations, symptoms, emotional function, and environmental stimuli. Individual questions are equally weighted. The overall AQLQ score is the mean of the responses to each of the 32 questions and ranges from 1 to 7. A score 7.0 indicates that the patient has no impairments due to asthma and score 1.0 indicates severe impairment.

Embodiments described herein are directed to methods of treating the inadequately controlled moderate to severe asthma of the eosinophilic phenotype in the subject comprising orally administering a daily dose of about 150 mg to about 300 mg of dexpramipexole, wherein the subject's quality of life is improved. The Asthma Quality of Life Questionnaire (AQLQ) can be used to assess an individual's quality of life, scores range from 1-7, with higher scores indicating better quality of life. In embodiments described herein, the inadequately controlled asthmatic human subject has a change in AQLQ score of at least 0.5 points. In embodiments described herein, the inadequately controlled asthmatic human subject has an improvement in score on Asthma Quality of Life Questionnaire (AQLQ) and the score is greater than 3.5.

In embodiments described herein, the inadequately controlled asthmatic human subject has an improvement in score on the St. George's Respiratory Questionnaire.

In some embodiments, treating the inadequately controlled moderate to severe asthma of the eosinophilic phenotype in the subject by administration of a daily dose of about 150 mg to about 300 mg of dexpramipexole results in improving a subject's overall morbidity. In some embodiments, treating the inadequately controlled moderate to severe asthma of the eosinophilic phenotype in the subject by administration of a daily dose of about 75 mg to about 300 mg of dexpramipexole results in improving a subject's overall mortality risk.

Surprisingly, treatment with dexpramipexole provides a remittive effect. Where treatment is discontinued following a predetermined period of treatment, the subject may continue to exhibit control of the severe asthma. In certain embodiments, the predetermined period of treatment is about 24 weeks. In additional embodiments, the predetermined period of treatment is about 20 weeks, about 16 weeks, about 12 weeks, about 8 weeks, or about 4 weeks. In certain embodiments, the moderate to severe asthma of the eosinophilic phenotype remains controlled after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. In certain embodiments, the level of blood eosinophils remains reduced after dexpramipexole has been discontinued for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks. Furthermore, where the subject restarts treatment following the discontinuation, the subject may exhibit a lack of tachyphylaxis, i.e., the subject responds to treatment in a substantially similar manner as prior to discontinuation (e.g., without a partial and/or progressive decrease in response).

In certain embodiments, the dexpramipexole is administered in combination with a bronchodilator selected from the group consisting of levalbuterol, ipratropium bromide, budesonide/formoterol, ipratropium, fluticasone/salmeterol, and combinations thereof. In certain embodiments, the dexpramipexole is administered in combination with an anti-inflammatory selected from the group consisting of zileuton, zafirlukast, and combinations thereof.

In certain embodiments, the dexpramipexole is administered instead of mepolizumab (NUCALA®). In certain embodiments, the dexpramipexole is administered instead of reslizumab (CINQAIR®). In certain embodiments, the dexpramipexole is administered instead of benralizumab (FASENRAR). In certain embodiments, the dexpramipexole is administered instead of dupilumab (DUPIXENT®). In certain embodiments, the dexpramipexole is administered instead of tezepelumab.

One of ordinary skill in the art will understand and appreciate the dosages and timing of the dosages to be administered to a human subject in need thereof. The doses and duration of treatment may vary, and may be based on assessment by one of ordinary skill in the art based on monitoring and measuring improvements in pulmonary and non-pulmonary tissues. This assessment may be made based on outward physical signs of improvement, such as decreased wheezing or less shortness of breath, or on internal physiological signs or markers. The doses may also depend on the condition or disease being treated, the degree of the condition or disease being treated and further on the age, weight, body mass index and body surface area of the subject.

In some embodiments, therapeutically effective amounts, or single unit doses of dexpramipexole may be administered multiple times per day, such as twice per day, i.e., BID. However, in additional embodiments, doses may be administered three times per day, four times per day, or five times per day.

Embodiments are also directed to a dosage regimen for orally administering dexpramipexole or a pharmaceutically acceptable salt thereof to treat the conditions disclosed herein. For example, in some embodiments, the methods described herein may comprise a dosage regimen that may include a plurality of daily doses having an equal amount of dexpramipexole or a pharmaceutically acceptable salt thereof as the initial dose in one or more unit doses. In other embodiments, the dosage regimen may include an initial dose of dexpramipexole or a pharmaceutically acceptable salt thereof in one or more unit doses, then a plurality of daily doses having a lower amount of dexpramipexole or a pharmaceutically acceptable salt thereof as the initial dose in one or more unit doses. The dosage regimen may administer an initial dose followed by one or more maintenance doses. The plurality of doses following the administering of an initial dose may be maintenance doses.

Such embodiments are not limited by the amount of the initial dose and daily doses. For example, in particular embodiments, the initial dose and each of the plurality of daily doses may be from about 150 mg to about 300 mg of dexpramipexole.

In some embodiments, two unit doses of about 75 mg are administered daily, wherein each unit dose may be substantially equal. In some embodiments, three unit doses totaling to about 150 mg are administered daily, wherein each unit dose may be substantially equal.

In some embodiments, two unit doses of about 150 mg are administered daily, wherein each unit dose may be substantially equal. In some embodiments, three unit doses totaling to about 300 mg are administered daily, wherein each unit dose may be substantially equal.

Following the initial dose for a period of time, the maintenance dose may include administering less than the initial daily dose of 150 mg to about 300 mg administered twice per day as a 37.5 mg, 75 mg, or 150 mg tablet, respectively, or administering the daily dose once per day. In preferred embodiments, the maintenance dose is 75 mg once per day, 150 mg, once per day, or 300 mg once per day.

In further embodiments, the method may include an initial dosing regimen and a maintenance dosing regimen. In certain embodiments, the initial dosing regimen may include administering a higher dose of dexpramipexole or a pharmaceutically acceptable salt thereof than the maintenance dosing regimen as either a single administration or by administering an increased dosage for a limited period of time prior to beginning a maintenance dosing regimen of dexpramipexole or a pharmaceutically acceptable salt thereof. In some embodiments, subjects undergoing a maintenance regimen may be administered one or more higher-dosage treatments at one or more times during the maintenance dosage regimen.

In some embodiments, the initial dosing regimen and the maintenance dosing regimen may include administering dexpramipexole or a pharmaceutically acceptable salt thereof once per day, or twice per day. In such embodiments, the dosage regimen may continue administering an initial dose for 1, 2, 3, 4, 5, 6 or 7 days, up to 4 weeks, up to 8 weeks or up to 12 weeks. In some embodiments, the dosage regimen for administering an initial dose and/or a maintenance dose may continue for an extended period of time. Various embodiments are directed to a dosing regimen for dexpramipexole or a pharmaceutically acceptable salt thereof in which maintenance doses are maintained for an extended period of time without titration or otherwise changing the dosing regimen. In such embodiments, the extended period of time may be about 12 weeks or longer, about 6 months or longer, about 1 year or longer, 2, 3, 4, 5, or 10 years or longer, and in certain embodiments, an indefinite period of time.

In some embodiments, treatment with a daily dose of about 150 mg to about 300 mg of dexpramipexole is without the adverse side effects associated with dopamine agonism.

Oral pharmaceutical compositions containing dexpramipexole or a pharmaceutically acceptable salt thereof in a solid dosage may include, but are not limited to, softgels, tablets, capsules, cachets, pellets, pills, powders and granules; other oral dosage forms include, but are not limited to, solutions, suspensions, emulsions, and dry powder.

The devices, systems, and methods as described herein are not intended to be limited in terms of the particular embodiments described, which are intended only as illustrations of various features. Many modifications and variations to the devices, systems, and methods can be made without departing from their spirit and scope, as will be apparent to those skilled in the art.

Additional information regarding treatment of asthma of the eosinophilic phenotype described herein can be found in International Patent Application No. PCT/US2021/044719 entitled “Use of Dexpramipexole for the Treatment of Moderate to Severe Asthma,” filed Aug. 5, 2021, which is hereby incorporated herein by reference in its entirety.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples:

### EXAMPLES

### Example 1: Description of Dosage Form

The tablets for oral administration containing 150 mg of dexpramipexole dihydrochloride monohydrate are formulated as described in Table A.

TABLE A
Composition of Dexpramipexole Tablets

| | | Quantity per |
|---|---|---|
| Component | Function | tablet (mg) |
| Dexpramipexole dihydrochloride | Drug Substance | 150.0 |
| monohydrate | | |
| Microcrystalline | Diluent | 132.2 |
| cellulose/Cellulose, | | |
| microcrystalline | | |
| Mannitol | Diluent | 75.0 |
| Crospovidone | Disintegrant | 15.0 |
| Magnesium stearate | Glidant | 2.8 |

| | |
|---|---|
| Core Total | 375.0 |

Tablet Coat

| | | |
|---|---|---|
| Opadry II white | Film former | 11.3 |

| | |
|---|---|
| Coated tablet Total | 386.3 |

Summary of Tablets Containing 12.5, 20, 37.5, 75, 125, 150, and 200 mg of dexpramipexole dihydrochloride monohydrate is provided in Table B. Tablets having 125 mg, 150 mg, or 200 mg comprise the same relative formulation, however the size of the tablet will be proportional to the dosage amount.

TABLE B
Formulations

| | Tablet Strength (mg) and Formulation (%) | | | | | |
|---|---|---|---|---|---|---|
| Component | 12.5 mg | 37.5 mg | 75 mg | 125 mg | 150 mg | 200 mg |
| Dexpramipexole | 4.00 | 12.00 | 24.00 | 40.00 | 40.00 | 40.00 |
| dihydrochloride | | | | | | |
| monohydrate (%) | | | | | | |
| Microcrystalline | 58.22 | 53.11 | 45.46 | 35.25 | 35.25 | 35.25 |
| cellulose/Cellulose, | | | | | | |
| microcrystalline (%) | | | | | | |
| Mannitol (%) | 33.03 | 30.14 | 25.79 | 20.00 | 20.00 | 20.00 |
| Crospovidone (%) | 4.00 | 4.00 | 4.00 | 4.00 | 4.00 | 4.00 |
| Magnesium stearate (%) | 0.75 | 0.75 | 0.75 | 0.75 | 0.75 | 0.75 |
| Total (%) | 100 | 100 | 100 | 100 | 100 | 100 |

### Example 2: Description of Dosage Form

The tablets for oral administration containing dexpramipexole dihydrochloride monohydrate at an amount equivalent to 150 mg of dexpramipexole dihydrochloride were formulated as described in Table C.

TABLE C
Composition of Dexpramipexole Tablets

| | | Quantity per |
|---|---|---|
| Component | Function | tablet (mg) |
| Dexpramipexole dihydrochloride | Drug Substance | 159.50* |
| monohydrate | | |
| Microcrystalline | Diluent | 132.19 |
| cellulose/Cellulose, | | |
| microcrystalline | | |
| Mannitol | Diluent | 65.50 |
| Crospovidone | Disintegrant | 15.00 |
| Magnesium stearate | Glidant | 2.81 |

| | |
|---|---|
| Core Total | 375.0 |

Tablet Coat

| | | |
|---|---|---|
| Opadry II white | Film former | 11.25 |

| | |
|---|---|
| Coated tablet Total | 386.25 |

*159.50 mg of dexpramipexole dihydrochloride monohydrate is equivalent to 150.0 mg dexpramipexole dihydrochloride based on molecular weight ratio of monohydrate to anhydrous API.

The tablets for oral administration containing dexpramipexole dihydrochloride monohydrate at an amount equivalent to 75 mg of dexpramipexole dihydrochloride were formulated as described in Table D.

TABLE D
Composition of Dexpramipexole Tablets

| | | Quantity per |
|---|---|---|
| Component | Function | tablet (mg) |
| Dexpramipexole dihydrochloride | Drug Substance | 79.75* |
| monohydrate | | |
| Microcrystalline | Diluent | 180.0 |
| cellulose/Cellulose, | | |
| microcrystalline | | |
| Mannitol | Diluent | 97.45 |
| Crospovidone | Disintegrant | 15.00 |
| Magnesium stearate | Glidant | 2.80 |

| | |
|---|---|
| Core Total | 375.0 |

Tablet Coat

| | | |
|---|---|---|
| Opadry II white | Film former | 11.25 |

| | |
|---|---|
| Coated tablet Total | 386.25 |

*79.75 mg of dexpramipexole dihydrochloride monohydrate is equivalent to 75 mg dexpramipexole dihydrochloride based on molecular weight ratio of monohydrate to anhydrous API.

The tablets for oral administration containing dexpramipexole dihydrochloride monohydrate at an amount equivalent to 75 mg (Table D) and 150 mg (Table C) of dexpramipexole dihydrochloride were formulated and tested in the Examples below.

### Example 3: A Randomized, Double-Blind, Placebo-Controlled, Parallel-Group Study to Assess the Efficacy, Safety, and Tolerability of Dexpramipexole Administered Orally for 52 Weeks in Participants with Severe Eosinophilic Asthma (EXHALE-2)

The AR-DEX-22-01 trial (summary set forth in Table E) will investigate the safety, tolerability, and efficacy of dexpramipexole in participants with inadequately controlled severe eosinophilic asthma. While findings have shown that dexpramipexole may be effective in moderate to severe asthma, causing significant, dose-dependent, and durable reductions of eosinophils in blood and airways and changes in pre-bronchodilator forced expiratory volume (FEV₁), this study will evaluate the ability of selected dose regiments to maintain clinically meaningful reduction in severe asthma exacerbations over a greater period of time (i.e., 52 weeks or more) in subjects with inadequately controlled severe eosinophilic asthma. The study will rely particularly on annualized rate of severe asthma exacerbations (AAER) over 52 weeks, pre-bronchodilator FEV₁, changes in ACQ-6 evaluation, and changes in AQLQ+12 evaluation.

TABLE E
Summary of Clinical Study Trial (EXHALE-2)

| Protocol number: | AR-DEX-22-01 | |
|---|---|---|
| Protocol Title | A randomized, double-blind, placebo-controlled, parallel-group study to | |
| | assess the efficacy, safety, and tolerability of dexpramipexole administered | |
| | orally for 52 weeks in participants with severe eosinophilic asthma | |
| | (EXHALE-2) | |
| Investigational | Dexpramipexole tablets for oral use | |
| Product: | | |
| Name of Active | Dexpramipexole dihydrochloride | |
| Ingredient: | | |
| Indication: | Severe eosinophilic asthma | |
| Phase of | III | |
| Development: | | |
| Study Rationale: | The results from the Phase II dose finding study with dexpramipexole in | |
| | moderate to severe asthma (EXHALE) include significant, dose-dependent, | |
| | and durable reductions of eosinophils in blood and airways. In addition, | |
| | clinically meaningful, placebo-corrected, change from baseline in pre- | |
| | bronchodilator (pre-BD) forced expiratory volume in 1 second (FEV1) were | |
| | observed across the dose groups at various timepoints. The safety and | |
| | tolerability profile of dexpramipexole in this Phase II study was similar to | |
| | placebo and supports advancing the program into Phase III. | |
| | The selected doses of 75 mg and 150 mg twice daily (BID) are supported | |
| | by the safety and efficacy data from the completed Phase II asthma study | |
| | (EXHALE) along with the safety data from the Phase II and III | |
| | amyotrophic lateral sclerosis studies. These doses have the potential to | |
| | maintain a clinically meaningful reduction in eosinophils, which should | |
| | lead to a significant reduction in severe asthma exacerbations. | |
| | The objective of this clinical study is to investigate the safety, tolerability, | |
| | and efficacy of dexpramipexole in participants with inadequately controlled | |
| | severe eosinophilic asthma. | |
| Study Design: | This is a Phase III, randomized, double-blind, placebo-controlled, parallel- | |
| | group, multicenter study. | |
| Study | Efficacy: | |
| Assessments: | Severe asthma exacerbations, asthma control, pulmonary function, and | |
| | quality of life. | |
| | Pharmacokinetics: | |
| | Plasma samples from each participant to measure drug concentrations, | |
| | drawn prior to and 2 hours post investigational drug dosing. | |
| | Biomarkers: | |
| | Blood eosinophil count. | |
| | Safety: | |
| | Physical examination; vital signs (systolic and diastolic blood pressure, | |
| | respiratory rate, pulse, and temperature); body weight; 12-lead | |
| | electrocardiogram (ECG); clinical laboratory evaluations (hematology, | |
| | blood chemistry, and urinalysis); pregnancy testing; adverse event (AE) | |
| | monitoring; and concomitant medication monitoring throughout the study. | |
| Study Objectives: | Primary Objective | |
| | To demonstrate the efficacy of dexpramipexole in reducing severe | |
| | asthma exacerbations. | |
| | Secondary Objectives | |
| | To demonstrate the efficacy of dexpramipexole on pulmonary function. | |
| | To demonstrate the efficacy of dexpramipexole on asthma control and | |
| | quality of life | |
| | To evaluate the effect of dexpramipexole on blood eosinophils. | |
| | Exploratory Objectives | |
| | To evaluate the kinetics of blood eosinophil lowering. | |
| | To examine the relationship between dexpramipexole exposure and | |
| | severe asthma exacerbations and other efficacy parameters. | |
| | Safety Objective | |
| | To evaluate the safety and tolerability of dexpramipexole administered | |
| | for 52 weeks in participants with eosinophilic asthma. | |
| Study Endpoints: | Primary Endpoint | |
| | Annualized rate of severe asthma exacerbations (AAER) over 52 | |
| | weeks. | |
| | Key Secondary Endpoints | |
| | Pre-BD FEV1, absolute change from baseline, averaged across visits at | |
| | Weeks 36, 44, and 52. | |
| | Asthma Control Questionnaire-6 (ACQ-6), change from baseline, | |
| | averaged across visits at Weeks 36, 44, and 52. | |
| | Standardized version of the Asthma Quality of Life Questionnaire for | |
| | 12 years and older (AQLQ + 12) change from baseline to Week 52. | |
| | Other Secondary Endpoints: | |
| | Annualized rate over 52 weeks of severe exacerbations requiring an | |
| | emergency department (ED) visit or hospitalization. | |
| | Annualized rate of severe asthma exacerbations from Week 4 to Week | |
| | 52. | |
| | AEC, change from baseline to Week 52 | |
| | Forced vital capacity (FVC), change from baseline, averaged over | |
| | Weeks 36, 44, and 52 | |
| | FVC, change from baseline at Weeks 4, 12, 20, 28, 36, 44, and 52. | |
| | Post-bronchodilator FEV₁, change from baseline to Week 52 | |
| | Peak expiratory flow (PEF), change from baseline to Week 52 | |
| | Time to first severe asthma exacerbation | |
| | Total asthma symptom score, change from baseline to Week 52 | |
| | The EuroQol five-dimensional questionnaire (EQ-5D-5L) change from | |
| | baseline to Week 52 | |
| | Exploratory Endpoints | |
| | Correlation between dexpramipexole exposure and severe asthma | |
| | exacerbations and other efficacy parameters. | |
| | Safety Endpoints | |
| | The safety endpoints of this study are measured by the following | |
| | assessments: physical examination; vital signs (systolic and diastolic blood | |
| | pressure, respiratory rate, pulse, and temperature); body weight; 12-lead | |
| | ECG; clinical laboratory evaluations (hematology, blood chemistry, and | |
| | urinalysis); pregnancy testing; AE monitoring; and concomitant medication | |
| | monitoring throughout the study. | |
| Study | The study will include participants with inadequately controlled severe | |
| Population: | asthma, aged ≥12 years, on Global Initiative for Asthma (GINA) 2021 Step | |
| | 4 or 5 therapy. | |
| Inclusion criteria: | To be eligible to participate in this study, candidates must meet the | |
| | following eligibility criteria at the Screening Visit or at the timepoint | |
| | specified in the individual eligibility criterion listed below: | |
| | 1. | Signed informed consent form and assent form, as appropriate. |
| | 2. | Male or female ≥12 years of age at randomization |
| | Asthma-related criteria | |
| | 3. | Documented physician diagnosis of asthma ≥12 months. |
| | 4. | Treatment of asthma, participants must satisfy all the below (items a |
| | to c) | |
| | a. | Participants who have received asthma controller medication |
| | with medium or high dose inhaled corticosteroids (IDS; ≥500 | |
| | ug/day fluticasone propionate dry powder formulation daily or | |
| | clinically comparable, per GINA 2021) on a regular basis for at | |
| | least 12 months prior to screening. Equivalent medium and high | |
| | dose ICS doses are detailed in Appendix C. | |
| | b. | Documented treatment with a stable dose of either medium or |
| | high dose ICS for at least 3 months prior to Visit 1. The ICS | |
| | may be contained within an ICS/long-acting β2 agonist (LABA) | |
| | combination product. As noted in Section 5.2.2, daily oral | |
| | corticosteroids are an allowed concomitant medication; | |
| | participants on daily oral corticosteroids must be on a stable | |
| | dose for 3 months before Screening Visit 1. | |
| | c. | Use of one of more additional daily maintenance asthma |
| | controller medications according to standard practice of care is | |
| | required; eg, LABA, leukotriene antagonist, theophylline, long- | |
| | acting muscarinic antagonists, cromolyn/nedocromil. Use of a | |
| | stable dose of any additional asthma controller medications must | |
| | be documented for at least 3 months prior to screening. | |
| | 5. | Pre BD FEV₁ ≥40% and <80% of predicted at Screening. |
| | 6. | Variable airflow obstruction documented with at least one of the |
| | following criteria: | |
| | a. | Bronchodiator reversibility during screening, as evidenced |
| | by ≥12% and ≥200 mL improvement in FEV1, 15 to 30 minutes | |
| | following inhalation of 400 μg (four puffs) of | |
| | albuterol/salbutamol (≥12% and ≥160 mL for ages 12 to 17). | |
| | Participants who do not meet the bronchodilator reversibility | |
| | inclusion criterion but have ≥10% and ≥160 mL reversibility, | |
| | may repeat the reversibility spirometry assessment once during | |
| | the Screening period, at an unscheduled visit prior to baseline. | |
| | b. | Bronchodilator reversibility, using the criteria above, |
| | documented in the past 12 months. | |
| | c. | Peak flow variation of ≥20% over a 2-week period, documented |
| | in the past 12 months. | |
| | d. | Airflow variability in clinic FEV₁ ≥20% between two |
| | consecutive clinic visits, documented in the past 12 months. | |
| | e. | Airway hyperresponsiveness (provocative concentration causing |
| | a 20% fall in FEV₁ of methacholine <8 mg/mL) documented in | |
| | the past 12 months. | |
| | 7. | ACQ-6 ≥1.5 at Screening. |
| | 8. | Documented history of at least two asthma exacerbations requiring |
| | treatment with systemic corticosteroids (intramuscular, intravenous, | |
| | or oral) within the past 12-month period. | |
| | General medical history | |
| | 9. | Negative urine pregnancy test for women of childbearing potential |
| | (WOCBP; after menarche) at Screening and Baseline. | |
| | 10. | WOCBP must use either of the following methods of birth control, |
| | from Screening through the End of Study Visit: | |
| | a. | A highly effective form of birth control (confirmed by the |
| | investigator). Highly effective forms of birth control include: | |
| | true sexual abstinence, a vasectomized sexual partner, Implanon, | |
| | female sterilization by tubal occlusion, any effective Intrauterine | |
| | device (IUD), IUC/intrauterine system (IUS), Levonorgestrel | |
| | Intrauterine system, or oral contraceptive | |
| | Or | |
| | b. | Two protocol acceptable methods of contraception in tandem. |
| | Women not of childbearing potential are defined as women who are | |
| | either permanently sterilized (hysterectomy, bilateral oophorectomy, | |
| | or bilateral salpingectomy), or who are postmenopausal. Women | |
| | will be considered postmenopausal if they have been amenorrheic | |
| | for ≥12 months prior to the planned date of randomization without | |
| | an alternative medical cause. The following age specific | |
| | requirements apply: | |
| | c. | Women <50 years old would be considered postmenopausal if |
| | they have been amenorrheic for 12 months or more following | |
| | cessation of exogenous hormonal treatment and follicle | |
| | stimulating hormone levels in the postmenopausal range. | |
| | d. | Women ≥50 years old would be considered postmenopausal if |
| | they have been amenorrheic for 12 months or more following | |
| | cessation of all exogenous hormonal treatment. | |
| Exclusion | Study participant candidates will be excluded from study entry if any of the | |
| criteria: | following exclusion criteria exist at the Screening Visit or at the timepoint | |
| | specified in the individual criterion listed below: | |
| | Asthma-related criteria: | |
| | 1. | A participant who experiences a severe asthma exacerbation |
| | (defined as a deterioration of asthma that results in emergency | |
| | treatment, hospitalization due to asthma, or treatment with systemic | |
| | corticosteroids) at any time from 4 weeks prior to the Screening | |
| | Visit up to and including the Baseline Visit. Participants who | |
| | experience an asthma exacerbation during the Screening/Run-in | |
| | Period may remain in screening and proceed with study visits 14 | |
| | days after they have completed their course of oral steroids or | |
| | returned to their pre-Screening Visit maintenance dose of oral | |
| | steroids and the investigator considers participant has returned to | |
| | baseline status. | |
| | 2. | Current diagnosis of diseases which may confound interpretation of |
| | this study's findings such as allergic bronchopulmonary | |
| | aspergillosis, eosinophilic granulomatosis with polyangiitis, | |
| | eosinophilic gastrointestinal diseases, hypereosinophilic syndrome, | |
| | chronic obstructive pulmonary disease, idiopathic pulmonary | |
| | fibrosis. | |
| | 3. | Respiratory infection: Upper or lower respiratory tract, sinus, or |
| | middle ear infection within the 4 weeks before Screening. | |
| | 4. | For participants aged 12 to 17 years old, AEC of <0.15 × 10⁹/L at |
| | Screening. | |
| | Prohibited medications/procedures | |
| | 5. | Treatment with a biologic investigational drug in the last 5 months. |
| | Treatment with non-biologic investigational drugs in the previous 30 | |
| | days or five-half-lives, whichever is longer. Treatment with | |
| | GSK3511294 (long-acting anti-IL-5) in the past 12 months. | |
| | 6. | Treatment with any of the following monoclonal antibody therapies |
| | within 120 days prior to Baseline: benralizumab, dupilumab, | |
| | mepolizumab, reslizumab, omalizumab, tezepelumab, or | |
| | tralokinumab. | |
| | 7. | Treatment with pramipexole (Mirapex ®) within 30 days of Baseline. |
| | 8. | Treatment with selected drugs known to have a substantial risk of |
| | neutropenia in the past 30 days (see Appendix A). | |
| | 9. | Bronchial thermoplasty procedure in the past 12 months or planned |
| | during the coming year. | |
| | General medical history | |
| | 10. | Weight <40 kg. |
| | 11. | Current smoking within the past year or a smoking history of >10 |
| | pack-years. Smoking includes tobacco, vaping, and/or marijuana | |
| | use. | |
| | 12. | Known or suspected alcohol or drug abuse |
| | 13. | Uncontrolled severer hypertension: systolic blood pressure >180 |
| | mmHg or diastolic blood pressure >110 mmHg prior to | |
| | randomization despite anti-hypertensive therapy. | |
| | 14. | History of malignancy that required surgery (excluding local and |
| | wide-local excision), radiation therapy and/or systemic therapy | |
| | during the 5 years prior to randomization. | |
| | 15. | History of human immunodeficiency virus (HIV) infection or |
| | chronic infection with hepatitis B or C. | |
| | 16. | A helminth parasitic infection diagnosed within 24 weeks prior to |
| | the date informed consent, and assent when applicable, that has not | |
| | been treated with or has failed to respond to standard of care (SoC) | |
| | therapy. | |
| | 17. | Medical or other condition likely to interfere with participant's |
| | ability to undergo study procedures, adhere to visit schedule, or | |
| | comply with study requirements. | |
| | 18. | Known or suspected noncompliance with medication. |
| | 19. | Unwillingness or inability to follow the procedures outlined in the |
| | protocol. | |
| | Clinical safety labs | |
| | 20. | Absolute neutrophil count <2.000 × 10⁹/L at screening. |
| | 21. | Renal dysfunction, defined as an estimated glomerular filtration rate |
| | (eGFR) <60 mL/min/1.73 m² at Screening (using the Chronic Kidney | |
| | Disease Epidemiology Collaboration [CKD-EPI] formula [Levy et | |
| | al, 2009] for age ≥18 years at screening; using the Bedside Schwartz | |
| | [Schwartz and Work, 2009] eGFR formula for age <18. | |
| | 22. | Active liver disease defined as any known current infections, |
| | neoplastic, or metabolic pathology of the liver or unexplained | |
| | elevations in alanine aminotransferase (ALT), aspartate | |
| | aminotransferase (AST), >3x the upper limit or normal (ULN), or | |
| | total bilirubin >2x ULN at screening confirmed by a repeat | |
| | abnormal measurement of the relevant value(s), at least 1 week | |
| | apart. | |
| | Cardiac safety | |
| | 23. | History of New York Heart Association class IV heart failure or last |
| | known left ventricular ejection fraction <25%. | |
| | 24. | History of major adverse cardiovascular event (MACE) within 3 |
| | months prior to randomization. | |
| | 25. | History of cardiac arrhythmia within 3 months prior to baseline that |
| | is not controlled by medication or via ablation. | |
| | 26. | History of long QT syndrome. |
| | 27. | Corrected QT interval by Fridericia (QTcF) interval >450 ms for |
| | males and >470 ms for females at Screening or QTcF ≥480 ms for | |
| | participants with bundle branch block. | |
| | 28. | Clinically important abnormalities in resting ECG that may interfere |
| | with the interpretation of QTcF interval changes at Screening, | |
| | including heart rate <45 beats per minute (bpm) or >100 bpm. | |
| | Pregnancy/Lactation | |
| | 29. | Pregnant women or women breastfeeding. |
| | 30. | Males who are unwilling to use an acceptable method of birth |

| | |
|---|---|
| | control during the entire study period (ie, condom with spermicide). |
| | For this study, rescreening may only be permitted under specific |
| | circumstances and only after contact with a Sponsor Clinical |
| | representative. Rescreening may be permitted, for example, for a |
| | participant who qualified for this study but did not enroll within the |
| | protocol prescribed screening period of 28 days due to logistical constraints |
| | or for administrative reasons. In addition, rescreening may be appropriate, |
| | at investigator and Sponsor discretion, following mild intercurrent illness |
| | after the condition has resolved. Otherwise, individuals who do not meet |
| | the eligibility criteria for participation in this study (screen failure) may not |
| | be rescreened. |
| | In case of rescreening, reconsent is required and all screening procedures |
| | must be repeated. The participant will be assigned a new screening number. |
| | It must be confirmed that participants meet all eligibility criteria under the |
| | new screening number and process before progressing to |
| | randomization/Day 1. |
| Study Period: | The estimated study period for this study will be approximately 2.75 years |
| | from first participant enrolled to last participant completed. |
| Duration of | The expected duration of each participant's involvement in the study will |
| Treatment and | be a maximum of 14 months (Screening through Follow-up Phase). |
| Follow-up: | Study Phases |
| | Screening Phase: Screening through initiation of Baseline. |
| | Participants will undergo screening to assess whether they satisfy |
| | the eligibility criteria. They will participate in the Run-in Phase (of |
| | up to 21 days duration, with a minimum of 14 days) as part of the |
| | Screening period, to confirm if their current asthma medication |
| | regimen is stable. |
| | Treatment Phase: Baseline through completion of Week 52. |
| | After the collection of all Baseline assessments, participants will |
| | begin investigational treatment (dexpramipexole 75 mg, 150 mg, or |
| | placebo), BID for 52 weeks. The Week 52 Visit is the Primary |
| | Outcome Visit for the study. |
| | Follow-up Phase: Conclusion of Week 52 assessments through Week 56. |
| | During this phase, participants will have one final phone visit to |
| | collect final safety assessments following cessation of |
| | investigational product. |
| | Participants may choose to continue to the long-term extension |
| | study. These participants will transition at Week 52 Visit and will |
| | not complete the Week 56 visit. |
| Investigational | Film coated tablets of dexpramipexole dihydrochloride (75 mg or 150 mg) |
| Product | or matching placebo, administered by mouth BID for 52 weeks |
| Administrations: | Dexpramipexole dihydrochloride 75 mg (dexpramipexole, 56 mg base |
| | equivalent) |
| | Dexpramipexole dihydrochloride 150 mg (dexpramipexole, 112 mg base |
| | equivalent) |

### Example 4: A Randomized, Double-Blind, Placebo-Controlled, Parallel-Group Study to Assess the Efficacy, Safety, and Tolerability of Dexpramipexole Administered Orally for 52 Weeks in Participants with Severe Eosinophilic Asthma (EXHALE-3)

The AR-DEX-22-02 trial (summary set forth in Table F) will investigate the safety, tolerability, and efficacy of dexpramipexole in participants with inadequately controlled severe eosinophilic asthma. While findings have shown that dexpramipexole may be effective in moderate to severe asthma, causing significant, dose-dependent, and durable reductions of eosinophils in blood and airways and changes in pre-bronchodilator forced expiratory volume (FEV₁), this study will evaluate the ability of selected dose regiments to maintain clinically meaningful reduction in severe asthma exacerbations over a greater period of time (i.e., 52 weeks or more) in subjects with inadequately controlled severe eosinophilic asthma, particularly where the subject has a blood eosinophil count (AEC) of ≥0.30×10⁹ cells/L. The study will rely particularly on annualized rate of severe asthma exacerbations (AAER) over 52 weeks, pre-bronchodilator FEV₁, changes in ACQ-6 evaluation, and changes in AQLQ+12 evaluation.

TABLE F
Summary of Clinical Study Trial (EXHALE-3)

| Protocol number: | AR-DEX-22-02 | |
|---|---|---|
| Protocol Title | A randomized, double-blind, placebo-controlled, parallel-group study to | |
| | assess the efficacy, safety, and tolerability of dexpramipexole administered | |
| | orally for 52 weeks in participants with severe eosinophilic asthma | |
| | (EXHALE-3) | |
| Investigational | Dexpramipexole tablets for oral use | |
| Product: | | |
| Name of Active | Dexpramipexole dihydrochloride | |
| Ingredient: | | |
| Indication: | Severe eosinophilic asthma | |
| Phase of | III | |
| Development: | | |
| Study Rationale: | The results from the Phase II dose finding study with dexpramipexole in | |
| | moderate to severe asthma (EXHALE) include significant, dose-dependent, | |
| | and durable reductions of eosinophils in blood and airways. In addition, | |
| | clinically meaningful, placebo-corrected, change from baseline in pre- | |
| | bronchodilator (pre-BD) forced expiratory volume in 1 second (FEV₁) were | |
| | observed across the dose groups at various timepoints. The safety and | |
| | tolerability profile of dexpramipexole in this Phase II study was similar to | |
| | placebo and supports advancing the program into Phase III. | |
| | The selected doses of 75 mg and 150 mg twice daily (BID) are supported | |
| | by the safety and efficacy data from the completed Phase II asthma study | |
| | (EXHALE) along with the safety data from the Phase II and III | |
| | amyotrophic lateral sclerosis studies. These doses have the potential to | |
| | maintain a clinically meaningful reduction in eosinophils, which should | |
| | lead to a significant reduction in severe asthma exacerbations. | |
| | The objective of this clinical study is to investigate the safety, tolerability, | |
| | and efficacy of dexpramipexole in participants with inadequately controlled | |
| | severe eosinophilic asthma. | |
| Study Design: | This is a Phase III, randomized, double-blind, placebo-controlled, parallel- | |
| | group, multicenter study. | |
| Study | Efficacy: | |
| Assessments: | Severe asthma exacerbations, asthma control, pulmonary function, and | |
| | quality of life. | |
| | Pharmacokinetics: | |
| | Plasma samples from each participant to measure drug concentrations, | |
| | drawn prior to and 2 hours post investigational drug dosing. | |
| | Biomarkers: | |
| | Blood eosinophil count. | |
| | Safety: | |
| | Physical examination; vital signs (systolic and diastolic blood pressure, | |
| | respiratory rate, pulse, and temperature); body weight; 12-lead | |
| | electrocardiogram (ECG); clinical laboratory evaluations (hematology, | |
| | blood chemistry, and urinalysis); pregnancy testing; adverse event (AE) | |
| | monitoring; and concomitant medication monitoring throughout the study. | |
| Study Objectives: | Primary Objective | |
| | To demonstrate the efficacy of dexpramipexole in reducing severe | |
| | asthma exacerbations. | |
| | Secondary Objectives | |
| | To demonstrate the efficacy of dexpramipexole on pulmonary function. | |
| | To demonstrate the efficacy of dexpramipexole on asthma control and | |
| | quality of life | |
| | To evaluate the effect of dexpramipexole on blood eosinophils. | |
| | Exploratory Objectives | |
| | To evaluate the kinetics of blood eosinophil lowering. | |
| | To examine the relationship between dexpramipexole exposure and | |
| | severe asthma exacerbations and other efficacy parameters. | |
| | Safety Objective | |
| | To evaluate the safety and tolerability of dexpramipexole administered | |
| | for 52 weeks in participants with eosinophilic asthma. | |
| Study Endpoints: | Primary Endpoint | |
| | Annualized rate of severe asthma exacerbations (AAER) over 52 | |
| | weeks. | |
| | Key Secondary Endpoints | |
| | Pre-BD FEV1, absolute change from baseline, averaged across visits at | |
| | Weeks 36, 44, and 52. | |
| | Asthma Control Questionnaire-6 (ACQ-6), change from baseline, | |
| | averaged across visits at Weeks 36, 44, and 52. | |
| | Standardized version of the Asthma Quality of Life Questionnaire for | |
| | 12 years and older (AQLQ + 12) change from baseline to Week 52. | |
| | Other Secondary Endpoints: | |
| | Annualized rate over 52 weeks of severe exacerbations requiring an | |
| | emergency department (ED) visit or hospitalization. | |
| | Annualized rate of severe asthma exacerbations from Week 4 to Week | |
| | 52. | |
| | AEC, change from baseline to Week 52 | |
| | Forced vital capacity (FVC), change from baseline, averaged over | |
| | Weeks 36, 44, and 52 | |
| | FVC, change from baseline at Weeks 4, 12, 20, 28, 36, 44, and 52. | |
| | Post-bronchodilator FEV1, change from baseline to Week 52 | |
| | Peak expiratory flow (PEF), change from baseline to Week 52 | |
| | Time to first severe asthma exacerbation | |
| | Total asthma symptom score, change from baseline to Week 52 | |
| | The EuroQol five-dimensional questionnaire (EQ-5D-5L) change from | |
| | baseline to Week 52 | |
| | Exploratory Endpoints | |
| | Correlation between dexpramipexole exposure and severe asthma | |
| | exacerbations and other efficacy parameters. | |
| | Safety Endpoints | |
| | The safety endpoints of this study are measured by the following | |
| | assessments: physical examination; vital signs (systolic and diastolic blood | |
| | pressure, respiratory rate, pulse, and temperature); body weight; 12-lead | |
| | ECG; clinical laboratory evaluations (hematology, blood chemistry, and | |
| | urinalysis); pregnancy testing; AE monitoring; and concomitant medication | |
| | monitoring throughout the study. | |
| Study | The study will include participants with inadequately controlled severe | |
| Population: | asthma, aged ≥12 years, on Global Initiative for Asthma (GINA) 2021 Step | |
| | 4 or 5 therapy with blood eosinophil count of ≥0.30 × 10⁹\L. | |
| Inclusion criteria: | To be eligible to participate in this study, candidates must meet the | |
| | following eligibility criteria at the Screening Visit or at the timepoint | |
| | specified in the individual eligibility criterion listed below: | |
| | 1. | Signed informed consent form and assent form, as appropriate. |
| | 2. | Male or female ≥12 years of age at randomization |
| | Asthma-related criteria | |
| | 3. | Documented physician diagnosis of asthma ≥12 months. |
| | 4. | Eosinophil count of ≥0.30 × 10⁹\L at Screening Visit 1. May be |
| | repeated at Screening Visit 2 if the initial value is between 0.250- | |
| | 0.299 × 10⁹\L. | |
| | 5. | Treatment of asthma, participants must satisfy all the below (items a |
| | to c) | |
| | a. | Participants who have received asthma controller medication |
| | with medium or high dose inhaled corticosteroids (IDS; ≥500 | |
| | μg/day fluticasone propionate dry powder formulation daily or | |
| | clinically comparable, per GINA 2021) on a regular basis for at | |
| | least 12 months prior to screening. Equivalent medium and high | |
| | dose ICS doses are detailed in Appendix C. | |
| | b. | Documented treatment with a stable dose of either medium or |
| | high dose ICS for at least 3 months prior to Visit 1. The ICS | |
| | may be contained within an ICS/long-acting β2 agonist (LABA) | |
| | combination product. As noted in Section 5.2.2, daily oral | |
| | corticosteroids are an allowed concomitant medication; | |
| | participants on daily oral corticosteroids must be on a stable | |
| | dose for 3 months before Screening Visit 1. | |
| | c. | Use of one of more additional daily maintenance asthma |
| | controller medications according to standard practice of care is | |
| | required; eg, LABA, leukotriene antagonist, theophylline, long- | |
| | acting muscarinic antagonists, cromolyn/nedocromil. Use of a | |
| | stable dose of any additional asthma controller medications must | |
| | be documented for at least 3 months prior to screening. | |
| | 6. | Pre BD FEV₁ ≥40% and <80% of predicted at Screening. |
| | 7. | Variable airflow obstruction documented with at least one of the |
| | following criteria: | |
| | a. | Bronchodiator reversibility during screening, as evidenced |
| | by ≥12% and ≥200 mL improvement in FEV₁, 15 to 30 minutes | |
| | following inhalation of 400 μg (four puffs) of | |
| | albuterol/salbutamol (≥12% and ≥160 mL for ages 12 to 17). | |
| | Participants who do not meet the bronchodilator reversibility | |
| | inclusion criterion but have ≥10% and ≥160 mL reversibility, | |
| | may repeat the reversibility spirometry assessment once during | |
| | the Screening period, at an unscheduled visit prior to baseline. | |
| | b. | Bronchodilator reversibility, using the criteria above, |
| | documented in the past 12 months. | |
| | c. | Peak flow variation of ≥20% over a 2-week period, documented |
| | in the past 12 months. | |
| | d. | Airflow variability in clinic FEV₁ ≥20% between two |
| | consecutive clinic visits, documented in the past 12 months. | |
| | e. | Airway hyperresponsiveness (provocative concentration causing |
| | a 20% fall in FEV₁ of methacholine <8 mg/mL) documented in | |
| | the past 12 months. | |
| | 8. | ACQ-6 ≥1.5 at Screening. |
| | 9. | Documented history of at least two asthma exacerbations requiring |
| | treatment with systemic corticosteroids (intramuscular, intravenous, | |
| | or oral) within the past 12-month period. | |
| | General medical history | |
| | 10. | Negative urine pregnancy test for women of childbearing potential |
| | (WOCBP; after menarche) at Screening and Baseline. | |
| | 11. | WOCBP must use either of the following methods of birth control, |
| | from Screening through the End of Study Visit: | |
| | a. | A highly effective form of birth control (confirmed by the |
| | investigator). Highly effective forms of birth control include: | |
| | true sexual abstinence, a vasectomized sexual partner, Implanon, | |
| | female sterilization by tubal occlusion, any effective Intrauterine | |
| | device (IUD), IUC/intrauterine system (IUS), Levonorgestrel | |
| | Intrauterine system, or oral contraceptive | |
| | Or | |
| | b. | Two protocol acceptable methods of contraception in tandem. |
| | Women not of childbearing potential are defined as women who are | |
| | either permanently sterilized (hysterectomy, bilateral oophorectomy, | |
| | or bilateral salpingectomy), or who are postmenopausal. Women | |
| | will be considered postmenopausal if they have been amenorrheic | |
| | for ≥12 months prior to the planned date of randomization without | |
| | an alternative medical cause. The following age specific | |
| | requirements apply: | |
| | c. | Women <50 years old would be considered postmenopausal if |
| | they have been amenorrheic for 12 months or more following | |
| | cessation of exogenous hormonal treatment and follicle | |
| | stimulating hormone levels in the postmenopausal range. | |
| | d. | Women ≥50 years old would be considered postmenopausal if |
| | they have been amenorrheic for 12 months or more following | |
| | cessation of all exogenous hormonal treatment. | |
| Exclusion | Study participant candidates will be excluded from study entry if any of the | |
| criteria: | following exclusion criteria exist at the Screening Visit or at the timepoint | |
| | specified in the individual criterion listed below: | |
| | Asthma-related criteria: | |
| | 1. | A participant who experiences a severe asthma exacerbation |
| | (defined as a deterioration of asthma that results in emergency | |
| | treatment, hospitalization due to asthma, or treatment with systemic | |
| | corticosteroids) at any time from 4 weeks prior to the Screening | |
| | Visit up to and including the Baseline Visit. Participants who | |
| | experience an asthma exacerbation during the Screening/Run-in | |
| | Period may remain in screening and proceed with study visits 14 | |
| | days after they have completed their course of oral steroids or | |
| | returned to their pre-Screening Visit maintenance dose of oral | |
| | steroids and the investigator considers participant has returned to | |
| | baseline status. | |
| | 2. | Current diagnosis of diseases which may confound interpretation of |
| | this study's findings such as allergic bronchopulmonary | |
| | aspergillosis, eosinophilic granulomatosis with polyangiitis, | |
| | eosinophilic gastrointestinal diseases, hypereosinophilic syndrome, | |
| | chronic obstructive pulmonary disease, idiopathic pulmonary | |
| | fibrosis. | |
| | 3. | Respiratory infection: Upper or lower respiratory tract, sinus, or |
| | middle ear infection within the 4 weeks before Screening. | |
| | Prohibited medications/procedures | |
| | 4. | Treatment with a biologic investigational drug in the last 5 months. |
| | Treatment with non-biologic investigational drugs in the previous 30 | |
| | days or five-half-lives, whichever is longer. Treatment with | |
| | GSK3511294 (long-acting anti-IL-5) in the past 12 months. | |
| | 5. | Treatment with any of the following monoclonal antibody therapies |
| | within 120 days prior to Baseline: benralizumab, dupilumab, | |
| | mepolizumab, reslizumab, omalizumab, tezepelumab, or | |
| | tralokinumab. | |
| | 6. | Treatment with pramipexole (Mirapex ®) within 30 days of Baseline. |
| | 7. | Treatment with selected drugs known to have a substantial risk of |
| | neutropenia in the past 30 days (see Appendix A). | |
| | 8. | Bronchial thermoplasty procedure in the past 12 months or planned |
| | during the coming year. | |
| | General medical history | |
| | 9. | Weight <40 kg. |
| | 10. | Current smoking within the past year or a smoking history of >10 |
| | pack-years. Smoking includes tobacco, vaping, and/or marijuana | |
| | use. | |
| | 11. | Known or suspected alcohol or drug abuse |
| | 12. | Uncontrolled severer hypertension: systolic blood pressure >180 |
| | mmHg or diastolic blood pressure >110 mmHg prior to | |
| | randomization despite anti-hypertensive therapy. | |
| | 13. | History of malignancy that required surgery (excluding local and |
| | wide-local excision), radiation therapy and/or systemic therapy | |
| | during the 5 years prior to randomization. | |
| | 14. | History of human immunodeficiency virus (HIV) infection or |
| | chronic infection with hepatitis B or C. | |
| | 15. | A helminth parasitic infection diagnosed within 24 weeks prior to |
| | the date informed consent, and assent when applicable, that has not | |
| | been treated with or has failed to respond to standard of care (SoC) | |
| | therapy. | |
| | 16. | Medical or other condition likely to interfere with participant's |
| | ability to undergo study procedures, adhere to visit schedule, or | |
| | comply with study requirements. | |
| | 17. | Known or suspected noncompliance with medication. |
| | 18. | Unwillingness or inability to follow the procedures outlined in the |
| | protocol. | |
| | Clinical safety labs | |
| | 19. | Absolute neutrophil count <2.000 × 10⁹/L at screening. |
| | 20. | Renal dysfunction, defined as an estimated glomerular filtration rate |
| | (eGFR) <60 mL/min/1.73 m² at Screening (using the Chronic Kidney | |
| | Disease Epidemiology Collaboration [CKD-EPI] formula [Levy et | |
| | al, 2009] for age ≥18 years at screening; using the Bedside Schwartz | |
| | [Schwartz and Work, 2009] eGFR formula for age <18. | |
| | 21. | Active liver disease defined as any known current infections, |
| | neoplastic, or metabolic pathology of the liver or unexplained | |
| | elevations in alanine aminotransferase (ALT), aspartate | |
| | aminotransferase (AST), >3x the upper limit or normal (ULN), or | |
| | total bilirubin >2x ULN at screening confirmed by a repeat | |
| | abnormal measurement of the relevant value(s), at least 1 week | |
| | apart. | |
| | Cardiac safety | |
| | 22. | History of New York Heart Association class IV heart failure or last |
| | known left ventricular ejection fraction <25%. | |
| | 23. | History of major adverse cardiovascular event (MACE) within 3 |
| | months prior to randomization. | |
| | 24. | History of cardiac arrhythmia within 3 months prior to baseline that |
| | is not controlled by medication or via ablation. | |
| | 25. | History of long QT syndrome. |
| | 26. | Corrected QT interval by Fridericia (QTcF) interval >450 ms for |
| | males and >470 ms for females at Screening or QTcF ≥480 ms for | |
| | participants with bundle branch block. | |
| | 27. | Clinically important abnormalities in resting ECG that may interfere |
| | with the interpretation of QTcF interval changes at Screening, | |
| | including heart rate <45 beats per minute (bpm) or >100 bpm. | |
| | Pregnancy/Lactation | |
| | 28. | Pregnant women or women breastfeeding. |
| | 29. | Males who are unwilling to use an acceptable method of birth |

| | |
|---|---|
| | control during the entire study period (ie, condom with spermicide). |
| | For this study, rescreening may only be permitted under specific |
| | circumstances and only after contact with a Sponsor Clinical |
| | representative. Rescreening may be permitted, for example, for a |
| | participant who qualified for this study but did not enroll within the |
| | protocol prescribed screening period of 28 days due to logistical constraints |
| | or for administrative reasons. In addition, rescreening may be appropriate, |
| | at investigator and Sponsor discretion, following mild intercurrent illness |
| | after the condition has resolved. Otherwise, individuals who do not meet |
| | the eligibility criteria for participation in this study (screen failure) may not |
| | be rescreened. |
| | In case of rescreening, reconsent is required and all screening procedures |
| | must be repeated. The participant will be assigned a new screening number. |
| | It must be confirmed that participants meet all eligibility criteria under the |
| | new screening number and process before progressing to |
| | randomization/Day 1. |
| Study Period: | The estimated study period for this study will be approximately 2.75 years |
| | from first participant enrolled to last participant completed. |
| Duration of | The expected duration of each participant's involvement in the study will |
| Treatment and | be a maximum of 14 months (Screening through Follow-up Phase). |
| Follow-up: | Study Phases |
| | Screening Phase: Screening through initiation of Baseline. |
| | Participants will undergo screening to assess whether they satisfy |
| | the eligibility criteria. They will participate in the Run-in Phase (of |
| | up to 21 days duration, with a minimum of 14 days) as part of the |
| | Screening period, to confirm if their current asthma medication |
| | regimen is stable. |
| | Treatment Phase: Baseline through completion of Week 52. |
| | After the collection of all Baseline assessments, participants will |
| | begin investigational treatment (dexpramipexole 75 mg, 150 mg, or |
| | placebo), BID for 52 weeks. The Week 52 Visit is the Primary |
| | Outcome Visit for the study. |
| | Follow-up Phase: Conclusion of Week 52 assessments through Week 56. |
| | During this phase, participants will have one final phone visit to |
| | collect final safety assessments following cessation of |
| | investigational product. |
| | Participants may choose to continue to the long-term extension |
| | study. These participants will transition at Week 52 Visit and will |
| | not complete the Week 56 visit. |
| Investigational | Film coated tablets of dexpramipexole dihydrochloride (75 mg or 150 mg) |
| Product | or matching placebo, administered by mouth BID for 52 weeks |
| Administrations: | Dexpramipexole dihydrochloride 75 mg (dexpramipexole, 56 mg base |
| | equivalent) |
| | Dexpramipexole dihydrochloride 150 mg (dexpramipexole, 112 mg base |
| | equivalent) |

### Example 5: A Randomized, Double-Blind, Placebo-Controlled, Parallel-Group Study to Assess the Efficacy, Safety, and Tolerability of Dexpramipexole Administered Orally for 24 Weeks in Participants with Eosinophilic Asthma (EXHALE-4)

The AR-DEX-22-03 trial (summary set forth in Table G) will investigate the safety, tolerability, and efficacy of dexpramipexole in participants with inadequately controlled moderate to severe eosinophilic asthma. While findings have shown that dexpramipexole may be effective in moderate to severe asthma, causing significant, dose-dependent, and durable reductions of eosinophils in blood and airways and changes in pre-bronchodilator forced expiratory volume (FEV₁), this study will evaluate the ability of selected dose regiments to maintain clinically meaningful reduction in severe asthma exacerbations over a greater period of time (i.e., 24 weeks or more) in subjects with inadequately controlled severe eosinophilic asthma, particularly where the subject has a blood eosinophil count (AEC) of ≥0.30×10⁹ cells/L. The study will rely particularly on pre-bronchodilator FEV₁, changes in ACQ-6 evaluation, and changes in AQLQ+12 evaluation.

TABLE G
Summary of Clinical Study Trial (EXHALE-4)

| Protocol number: | AR-DEX-22-03 | |
|---|---|---|
| Protocol Title | A randomized, double-blind, placebo-controlled, parallel-group study to | |
| | assess the efficacy, safety, and tolerability of dexpramipexole administered | |
| | orally for 24 weeks in participants with eosinophilic asthma (EXHALE-4) | |
| Investigational | Dexpramipexole tablets for oral use | |
| Product: | | |
| Name of Active | Dexpramipexole dihydrochloride | |
| Ingredient: | | |
| Indication: | Severe eosinophilic asthma | |
| Phase of | III | |
| Development: | | |
| Study Rationale: | Dexpramipexole has been shown to reduce absolute eosinophil count | |
| | (AEC) and increase forced expiratory volume in 1 second (FEV₁) in | |
| | participants with eosinophilic asthma. The purpose of this study is to | |
| | evaluate dexpramipexole as an add-on therapy in inadequately controlled | |
| | Global Initiative for Asthma (GINA) Step 3, 4, 5 asthma participants to | |
| | confirm improvements in lung function, asthma control, and quality of life. | |
| | In addition, the study will further evaluate the safety and tolerability of | |
| | dexpramipexole in participants with eosinophilic asthma. | |
| Study Design: | This is a Phase III, randomized, double-blind, placebo-controlled, parallel- | |
| | group, multicenter study. | |
| Study | Efficacy: | |
| Assessments: | Aasthma control, pulmonary function, and quality of life. | |
| | Pharmacokinetics: | |
| | Plasma samples will be drawn from all participants to measure drug | |
| | concentrations. Samples will be drawn prior to dosing with investigational | |
| | drug, and 2 hours post dose. | |
| | In addition, a PK substudy will be conducted to assist in the | |
| | characterization of dexpramipexole PK in participants (n = 30) with | |
| | eosinophilic asthma, including adolescents. Plasma samples will be drawn | |
| | at predose, 1, 2, 4, 6, and 8 hours post dosing from this subgroup of | |
| | participants on Day 1 and Week 4, and then prior to and 2 hours post | |
| | dosing at Week 12 and Week 24. Plasma will be analyzed to measure drug | |
| | concentrations. | |
| | Biomarkers: | |
| | Blood eosinophil count. | |
| | Safety: | |
| | Physical examination; vital signs (systolic and diastolic blood pressure, | |
| | respiratory rate, pulse, and temperature); body weight; 12-lead | |
| | electrocardiogram (ECG); clinical laboratory evaluations (hematology, | |
| | blood chemistry, and urinalysis); pregnancy testing; adverse event (AE) | |
| | monitoring; and concomitant medication monitoring throughout the study. | |
| Study Objectives: | Primary Objective | |
| | To evaluate the efficacy of dexpramipexole on pulmonary function. | |
| | Secondary Objectives | |
| | To evaluate the efficacy of dexpramipexole on asthma control, asthma | |
| | symptoms, and quality of life. | |
| | To evaluate the effect of dexpramipexole on blood eosinophils. | |
| | Exploratory Objectives | |
| | To examine the efficacy of dexpramipexole in reducing severe asthma | |
| | exacerbations. | |
| | To explore the relationship between blood eosinophil lowering and | |
| | other efficacy parameters. | |
| | To assess PK of dexpramipexole in adults and adolescents. | |
| | To explore the relationship between dexpramipexole exposure and | |
| | eosinophil lowering or other efficacy parameters, and dexpramipexole | |
| | exposure and selected safety parameters. | |
| | Safety Objective | |
| | To evaluate the safety and tolerability of dexpramipexole administered | |
| | for 24 weeks in participants with eosinophilic asthma. | |
| Study Endpoints: | Primary Endpoint | |
| | Pre-bronchodilator (pre-BD) FEV₁, absolute change from baseline, | |
| | averaged over Weeks 20 and 24. | |
| | Key Secondary Endpoints | |
| | Change from baseline in Asthma Control Questionnaire-6 (ACQ-6) | |
| | averaged across visits at Weeks 20 and 24. | |
| | Standardized version of the Asthma Quality of Life Questionnaire for | |
| | 12 years and older (AQLQ + 12) change from baseline to Week 24. | |
| | Other Secondary Endpoints: | |
| | Change from baseline AEC to Week 24. | |
| | Change from baseline forced vital capacity (FVC) averaged over | |
| | Weeks 20 and 24. | |
| | Change from baseline FVC at Weeks 4, 8, 12, 16, 20, and 24. | |
| | Change from baseline post-bronchodilator (post-BD) FEV₁ averaged | |
| | over Weeks 20 to 24. | |
| | Absolute change from baseline pre-BD FEV₁ at Weeks 4, 8, 12, 16, 20, | |
| | and 24. | |
| | The EuroQol five-dimensional questionnaire (EQ-5D-5L) change from | |
| | baseline to Week 24 | |
| | Exploratory Endpoints | |
| | PK parameters, such as maximum plasma concentration (Cₘₐₓ), time of | |
| | maximum plasma concentration (Tₘₐₓ), area under the curve to the end | |
| | of dosing (AUC_{0τ}), and trough concentration, will be determined from | |
| | plasma dexpramipexole concentrations and analyzed by population PK | |
| | modeling with a covariate analysis | |
| | Annualized rate of severe asthma exacerbations (AAER) over 24 | |
| | weeks. | |
| | Safety Endpoints | |
| | The safety endpoints of this study are measured by the following | |
| | assessments: physical examination; vital signs (systolic and diastolic blood | |
| | pressure, respiratory rate, pulse, and temperature); body weight; 12-lead | |
| | ECG; clinical laboratory evaluations (hematology, blood chemistry, and | |
| | urinalysis); pregnancy testing; AE monitoring; and concomitant medication | |
| | monitoring throughout the study. | |
| Study | The study will include participants with inadequately controlled asthma, | |
| Population: | aged ≥12 years, on GINA 2021 Step 3, 4 or 5 therapy | |
| Inclusion criteria: | To be eligible to participate in this study, candidates must meet the | |
| | following eligibility criteria at the Screening Visit or at the time point | |
| | specified in the individual eligibility criterion listed below: | |
| | 1. | Signed informed consent form and assent form, as appropriate. |
| | 2. | Male or female ≥12 years of age at randomization |
| | Asthma-related criteria | |
| | 3. | Documented physician diagnosis of asthma ≥12 months. |
| | 4. | Participants requiring at a minimum daily low dose inhaled |
| | corticosteroids (ICS; ≥100 μg/day fluticasone propionate dry | |
| | powder formulation daily or clinically comparable, per GINA 2021), | |
| | plus one or more additional daily maintenance asthma controller | |
| | medications, eg, long-acting β2 agonist (LABA) , leukotriene | |
| | antagonist, theophylline, long-acting muscarinic antagonists, | |
| | cromolyn/nedocromil. Use of daily ICS must be for at least 12 | |
| | weeks prior to Screening Visit 1 and the doses of all controller | |
| | medications must be stable for at least 4 weeks prior to Screening | |
| | Visit 1. | |
| | The ICS may be contained within an ICS/LABA combination | |
| | product. As noted in Section 5.2.2, daily oral corticosteroids are an | |
| | allowed concomitant medication. | |
| | 5. | Pre-BD FEV₁ ≥40% and <80% of predicted at Screening. |
| | 6. | Bronchodilator reversibility during screening, as evidenced by ≥12% |
| | and ≥200 mL improvement in FEV₁, 15 to 30 minutes following | |
| | inhalation of 400 μg (four puffs) of albuterol/salbutamol (≥12% | |
| | and ≥160 mL for ages 12 to 17). Participants who do not meet the | |
| | bronchodilator reversibility inclusion criterion but have ≥10% | |
| | and ≥160 mL reversibility, may repeat the reversibility spirometry | |
| | assessment once during the Screening Period, at an unscheduled | |
| | visit prior to baseline. | |
| | 7. | ACQ-6 ≥1.5 at Screening. |
| | 8. | Eosinophil count of ≥0.30 × 10⁹\L at Screening Visit 1. May be |
| | repeated prior to or at Screening Visit 2 if the initial value is | |
| | between 0.250 × 10⁹\L to 0.299 × 10⁹\L | |
| | General medical history | |
| | 9. | Negative urine pregnancy test for women of childbearing potential |
| | (WOCBP; after menarche) at Screening and Baseline. | |
| | 10. | WOCBP must use either of the following methods of birth control, |
| | from Screening through the End of Study Visit: | |
| | a. | A highly effective form of birth control (confirmed by the |
| | investigator). Highly effective forms of birth control include: | |
| | true sexual abstinence, a vasectomized sexual partner, Implanon, | |
| | female sterilization by tubal occlusion, any effective Intrauterine | |
| | device (IUD), IUC/intrauterine system (IUS), Levonorgestrel | |
| | Intrauterine system, or oral contraceptive | |
| | Or | |
| | b. | Two protocol acceptable methods of contraception in tandem. |
| | Women not of childbearing potential are defined as women who are | |
| | either permanently sterilized (hysterectomy, bilateral oophorectomy, | |
| | or bilateral salpingectomy), or who are postmenopausal. Women | |
| | will be considered postmenopausal if they have been amenorrheic | |
| | for ≥12 months prior to the planned date of randomization without | |
| | an alternative medical cause. The following age specific | |
| | requirements apply: | |
| | c. | Women <50 years old would be considered postmenopausal if |
| | they have been amenorrheic for 12 months or more following | |
| | cessation of exogenous hormonal treatment and follicle | |
| | stimulating hormone levels in the postmenopausal range. | |
| | d. | Women ≥50 years old would be considered postmenopausal if |
| | they have been amenorrheic for 12 months or more following | |
| | cessation of all exogenous hormonal treatment. | |
| Exclusion | Study participant candidates will be excluded from study entry if any of the | |
| criteria: | following exclusion criteria exist at the Screening Visit or at the timepoint | |
| | specified in the individual criterion listed below: | |
| | Asthma-related criteria: | |
| | 1. | A participant who experiences a severe asthma exacerbation |
| | (defined as a deterioration of asthma that results in emergency | |
| | treatment, hospitalization due to asthma, or treatment with systemic | |
| | steroids) at any time from 4 weeks prior to the Screening Visit up to | |
| | and including the Baseline Visit. Participants who experience an | |
| | asthma exacerbation during the Screening/Run-in Period may | |
| | remain in screening and proceed with study visits 14 days after they | |
| | have completed their course of oral steroids or returned to their pre- | |
| | Screening visit maintenance dose of oral steroids and the | |
| | investigator considers participant has returned to baseline status. | |
| | 2. | Current diagnosis of diseases which may confound interpretation of |
| | this study's findings such as allergic bronchopulmonary | |
| | aspergillosis, eosinophilic granulomatosis with polyangiitis, | |
| | eosinophilic gastrointestinal diseases, hypereosinophilic syndrome, | |
| | chronic obstructive pulmonary disease, idiopathic pulmonary | |
| | fibrosis. | |
| | 3. | Respiratory infection: Upper or lower respiratory tract, sinus, or |
| | middle ear infection within the 4 weeks before Screening. | |
| | Prohibited medications/procedures | |
| | 4. | Treatment with a biologic investigational drug in the last 5 months. |
| | Treatment with non-biologic investigational drugs in the previous 30 | |
| | days or five-half-lives, whichever is longer. Treatment with | |
| | GSK3511294 (long-acting anti-interleukin-5) in the past 12 months. | |
| | 5. | Treatment with any of the following monoclonal antibody therapies |
| | within 120 days prior to Baseline: benralizumab, dupilumab, | |
| | mepolizumab, reslizumab, omalizumab, tezepelumab, or | |
| | tralokinumab. | |
| | 6. | Treatment with pramipexole (Mirapex ®) within 30 days of Baseline. |
| | 7. | Treatment with selected drugs known to have a substantial risk of |
| | neutropenia in the past 30 days (see Appendix A). | |
| | 8. | Bronchial thermoplasty procedure in the past 12 months or planned |
| | during the coming year. | |
| | General medical history | |
| | 9. | Weight <40 kg. |
| | 10. | Current smoking within the past year or a smoking history of >10 |
| | pack-years. Smoking includes tobacco, vaping, and/or marijuana | |
| | use. | |
| | 11. | Known or suspected alcohol or drug abuse |
| | 12. | Uncontrolled severer hypertension: systolic blood pressure >180 |
| | mmHg or diastolic blood pressure >110 mmHg prior to | |
| | randomization despite anti-hypertensive therapy. | |
| | 13. | History of malignancy that required surgery (excluding local and |
| | wide-local excision), radiation therapy and/or systemic therapy | |
| | during the 5 years prior to randomization. | |
| | 14. | History of human immunodeficiency virus (HIV) infection or |
| | chronic infection with hepatitis B or C. | |
| | 15. | A helminth parasitic infection diagnosed within 24 weeks prior to |
| | the date informed consent, and assent when applicable, that has not | |
| | been treated with or has failed to respond to standard of care | |
| | therapy. | |
| | 16. | Medical or other condition likely to interfere with participant's |
| | ability to undergo study procedures, adhere to visit schedule, or | |
| | comply with study requirements. | |
| | 17. | Known or suspected noncompliance with medication. |
| | 18. | Unwillingness or inability to follow the procedures outlined in the |
| | protocol. | |
| | Clinical safety labs | |
| | 19. | Absolute neutrophil count <2.000 × 10⁹/L at Screening. |
| | 20. | Renal dysfunction, defined as an estimated glomerular filtration rate |
| | (eGFR) <60 mL/min/1.73 m² at Screening (using the Chronic Kidney | |
| | Disease Epidemiology Collaboration [CKD-EPI] formula [Levy et | |
| | al, 2009] for age ≥18 years at screening; using the Bedside Schwartz | |
| | [Schwartz and Work, 2009] eGFR formula for age <18. | |
| | 21. | Active liver disease defined as any known current infections, |
| | neoplastic, or metabolic pathology of the liver or unexplained | |
| | elevations in alanine aminotransferase (ALT), aspartate | |
| | aminotransferase (AST), >3x the upper limit or normal (ULN), or | |
| | total bilirubin >2x ULN at screening confirmed by a repeat | |
| | abnormal measurement of the relevant value(s), at least 1 week | |
| | apart. | |
| | Cardiac safety | |
| | 22. | History of New York Heart Association class IV heart failure or last |
| | known left ventricular ejection fraction <25%. | |
| | 23. | History of major adverse cardiovascular event (MACE) within 3 |
| | months prior to randomization. | |
| | 24. | History of cardiac arrhythmia within 3 months prior to baseline that |
| | is not controlled by medication or via ablation. | |
| | 25. | History of long QT syndrome. |
| | 26. | Corrected QT interval by Fridericia (QTcF) interval >450 ms for |
| | males and >470 ms for females at Screening or QTcF ≥480 ms for | |
| | participants with bundle branch block. | |
| | 27. | Clinically important abnormalities in resting ECG that may interfere |
| | with the interpretation of QTcF interval changes at Screening, | |
| | including heart rate <45 beats per minute (bpm) or >100 bpm. | |
| | Pregnancy/Lactation | |
| | 28. | Pregnant women or women breastfeeding. |
| | 29. | Males who are unwilling to use an acceptable method of birth |

| | |
|---|---|
| | control during the entire study period (ie, condom with spermicide). |
| | For this study, rescreening may only be permitted under specific |
| | circumstances and only after contact with a Sponsor Clinical |
| | Representative. |
| | This may be permitted, for example, for a participant who qualified for this |
| | study but did not enroll within the protocol prescribed screening period of |
| | 28 days due to logistical constraints or for administrative reasons. In |
| | addition, rescreening may be appropriate, at investigator and Sponsor |
| | discretion, following mild intercurrent illness after the condition has |
| | resolved. Otherwise, individuals who do not meet the eligibility criteria for |
| | participation in this study (screen failure) may not be rescreened. |
| | In case of rescreening, reconsent is required and all screening procedures |
| | must be repeated. The participant will be assigned a new screening number. |
| | It must be confirmed that participants meet all eligibility criteria under the |
| | new screening number and process before progressing to |
| | randomization/Day 1. |
| Study Period: | The estimated study period for this study will be approximately 1.5 years |
| | from first participant enrolled to last participant completed. |
| Duration of | The expected duration of each participant's involvement in the study will |
| Treatment and | be a maximum of 8 months (Screening through end of study). |
| Follow-up: | Study Phases |
| | Screening Phase: Screening/Run-in through initiation of Baseline. |
| | Screening Visit 1: Participants will undergo screening to assess |
| | whether they satisfy the eligibility criteria. |
| | Single-blind placebo Run-in Phase: Screening Visit 2 through |
| | initiation of Baseline |
| | Participants that continue to meet inclusion criteria at Screening |
| | Visit 2, will begin twice a day (BID) single-blind placebo tablets |
| | until the Baseline Visit (28 ± 3 days). At the end of the Run-in, |
| | participants who continue to meet study entry criteria will be |
| | randomized at the Baseline Visit. |
| | Treatment Phase: Baseline through completion of Week 24. |
| | After the collection of all Baseline assessments, participants will |
| | begin treatment with investigational product (dexpramipexole 75 |
| | mg, 150 mg, or placebo), BID for 24 weeks. The Week 24 Visit is |
| | the Primary Outcome Visit for the study. |
| | Follow-up Phase: Conclusion of Week 24 assessments through Week 28 |
| | During this phase, participants will have one final phone visit to |
| | collect final safety assessments following cessation of |
| | investigational product |
| Investigational | Film coated tablets of dexpramipexole dihydrochloride (75 mg or 150 mg) |
| Product | or matching placebo, administered by mouth BID for 24 weeks. |
| Administrations: | Dexpramipexole dihydrochloride 75 mg (dexpramipexole, 56 mg base |
| | equivalent) |
| | Dexpramipexole dihydrochloride 150 mg (dexpramipexole, 112 mg base |
| | equivalent) |

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the FIGURES, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method of treating inadequately controlled severe asthma of the eosinophilic phenotype in a human subject in need thereof comprising orally administering to the subject a daily dose of about 150 mg to about 300 mg of dexpramipexole, or a pharmaceutically acceptable salt thereof, wherein the subject is already receiving at least two asthma medications, thereby treating the inadequately controlled severe asthma of the eosinophilic phenotype in the subject,
wherein the at least two asthma medications comprise an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

2. The method of claim 1, wherein the daily dose of dexpramipexole, or a pharmaceutically acceptable salt thereof, is about 150 mg per day.

3. The method of claim 2, wherein the daily dose of dexpramipexole, or a pharmaceutically acceptable salt thereof, is administered as 75 mg twice daily.

4. The method of claim 1, wherein the daily dose of dexpramipexole, or a pharmaceutically acceptable salt thereof, is about 300 mg per day.

5. The method of claim 4, wherein the daily dose of dexpramipexole, or a pharmaceutically acceptable salt thereof, is administered as 150 mg twice daily.

6. The method of claim 1, wherein the daily dose of about 150 mg to about 300 mg of dexpramipexole is administered to the subject for up to 52 weeks.

7. The method of claim 1, wherein the subject is defined under Global Initiative for Asthma (GINA) Steps 4 and 5.

8. The method of claim 1, wherein the subject has a blood eosinophil count (AEC) of ≥0.30×10⁹ cells/L.

9. The method of claim 1, wherein treating the moderate to severe asthma of the eosinophilic phenotype in the subject comprises reducing a frequency of asthma exacerbations.

10. The method of claim 9, wherein the frequency of asthma exacerbations is assessed according to an annualized rate of severe asthma exacerbations (AAER).

11. The method of claim 9, wherein the frequency of asthma exacerbations is assessed according to a time between subsequent severe asthma exacerbations.

12. The method of claim 1, wherein treating the inadequately controlled severe asthma of the eosinophilic phenotype in the subject comprises improving a measurement selected from the group consisting of: pre-bronchodilator forced expiratory volume in 1 second (FEV₁), score on Asthma Control Questionnaire (ACQ), score on Asthma Quality of Life Questionnaire (AQLQ), and combinations thereof.

13. The method of claim 1, wherein treating the moderate to severe asthma of the eosinophilic phenotype in the subject comprises improving a measurement selected from the group consisting of: annualized rate of severe asthma exacerbations (AAER) requiring hospitalization or an emergency department visit, forced vital capacity (FVC), post-bronchodilator forced expiratory volume in 1 second (FEV₁), peak expiratory flow (PEF), time to first exacerbation, total asthma symptom score, EuroQol five-dimensional questionnaire (EQ-5D-5L), and combinations thereof.

14. The method of claim 1, wherein treating the moderate to severe asthma of the eosinophilic phenotype in the subject comprises reducing a level of absolute blood eosinophils of the subject.

15. The method of claim 14, wherein the level of absolute blood eosinophils is reduced by at least about 50%.

16. The method of claim 1, wherein the subject is greater than 12 years of age and less than 75 years of age.

17. The method of claim 1, wherein the subject is about 12 years of age to about 17 years of age.

18. The method of claim 1, wherein the subject is about 18 years of age or older.

19. The method of claim 1, wherein the inhaled corticosteroid (ICS) is selected from the group consisting of beclomethasone, fluticasone, ciclesonide, mometasone, budesonide, flunisolide, and combinations thereof.

20. The method of claim 1, wherein the long-acting beta agonist (LABA) is selected from the group consisting of albuterol sulfate, formoterol fumarate, salmeterol, salmeterol xinafoate, arformoterol tartrate, olodaterol, umeclidinium, vilanterol, indacaterol, and combinations thereof.

21. A method of treating inadequately controlled moderate to severe asthma of the eosinophilic phenotype in a human subject in need thereof comprising orally administering to the subject a daily dose of about 150 mg to about 300 mg of dexpramipexole, or a pharmaceutically acceptable salt thereof, wherein the subject is already receiving at least two asthma medications, thereby treating the inadequately controlled moderate to severe asthma of the eosinophilic phenotype in the subject,
wherein the at least two asthma medications comprise an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

22. The method of claim 21, wherein the daily dose of dexpramipexole, or a pharmaceutically acceptable salt thereof, is about 150 mg per day.

23. The method of claim 22, wherein the daily dose of dexpramipexole, or a pharmaceutically acceptable salt thereof, is administered as 75 mg twice daily.

24. The method of claim 21, wherein the daily dose of dexpramipexole, or a pharmaceutically acceptable salt thereof, is about 300 mg per day.

25. The method of claim 24, wherein the daily dose of dexpramipexole, or a pharmaceutically acceptable salt thereof, is administered as 150 mg twice daily.

26. The method of claim 21, wherein the daily dose of about 150 mg to about 300 mg of dexpramipexole is administered to the subject for up to 24 weeks.

27. The method of claim 21, wherein the subject is defined under Global Initiative for Asthma (GINA) Steps 3, 4 and 5.

28. The method of claim 21, wherein the subject has an absolute eosinophil count (AEC) in the blood of ≥0.30×10⁹ cells/L.

29. The method of claim 21, wherein treating the moderate to severe asthma of the eosinophilic phenotype in the subject comprises improving pulmonary function of the subject.

30. The method of claim 29, wherein the pulmonary function of the subject is assessed according to pre-bronchodilator forced expiratory volume in 1 second (FEV₁).

31. The method of claim 21, wherein treating the inadequately controlled severe asthma of the eosinophilic phenotype in the subject comprises improving a measurement selected from the group consisting of: score on Asthma Control Questionnaire (ACQ), score on Asthma Quality of Life Questionnaire (AQLQ), and combinations thereof.

32. The method of claim 21, wherein treating the moderate to severe asthma of the eosinophilic phenotype in the subject comprises improving a measurement selected from the group consisting of: an absolute eosinophil count (AEC), forced vital capacity (FVC), post-bronchodilator forced expiratory volume in 1 second (FEV₁), EuroQol five-dimensional questionnaire (EQ-5D-5L), and combinations thereof.

33. The method of claim 21, wherein treating the moderate to severe asthma of the eosinophilic phenotype in the subject comprises improving a measurement selected from the group consisting of: annualized rate of severe asthma exacerbations (AAER) over 24 weeks, pharmacokinetic parameters including but not limited to maximum plasma concentration (Cₘₐₓ), time of maximum plasma concentration (Tₘₐₓ), area under the curve to the end of dosing (AUC₀ₜ), and trough concentration, and combinations thereof.

34. The method of claim 21, wherein treating the moderate to severe asthma of the eosinophilic phenotype in the subject comprises reducing a level of absolute blood eosinophils of the subject.

35. The method of claim 34, wherein the level of absolute blood eosinophils is reduced by at least about 50%.

36. The method of claim 21, wherein the subject is greater than or equal to 12 years of age and less than 75 years of age.

37. The method of claim 21, wherein the subject is about 12 years of age to about 17 years of age.

38. The method of claim 21, wherein the subject is about 18 years of age or older.

39. The method of claim 21, wherein the inhaled corticosteroid (ICS) is selected from the group consisting of beclomethasone, fluticasone, ciclesonide, mometasone, budesonide, flunisolide, and combinations thereof.

40. The method of claim 21, wherein the long-acting beta agonist (LABA) is selected from the group consisting of albuterol sulfate, formoterol fumarate, salmeterol, salmeterol xinafoate, arformoterol tartrate, olodaterol, umeclidinium, vilanterol, indacaterol, and combinations thereof.

41. The method of claim 1, wherein the subject is already receiving a medium dose of the ICS on a regular basis for at least 12 months and on a stable dose for at least 3 months.

42. The method of claim 1, wherein the subject is already receiving the additional daily maintenance asthma medication for at least 3 months.

43. The method of claim 21, wherein the subject is already receiving a medium dose of the ICS on a regular basis for at least 12 months and on a stable dose for at least 3 months.

44. The method of claim 21, wherein the subject is already receiving the additional daily maintenance asthma medication for at least 3 months.

45. A method of treating inadequately controlled severe asthma of the eosinophilic phenotype in a human subject in need thereof comprising orally administering to the subject a daily dose of about 150 mg to about 300 mg of dexpramipexole, or a pharmaceutically acceptable salt thereof, wherein orally administering the daily dose reduces a frequency of asthma exacerbations in the subject, thereby treating the inadequately controlled severe asthma of the eosinophilic phenotype in the subject,
wherein the subject is already receiving at least two asthma medications comprising an inhaled corticosteroid (ICS) and an additional daily maintenance asthma medication selected from the group consisting of: a long-acting β2 agonist (LABA), a leukotriene antagonist, theophylline, a long-action muscarinic antagonist, and cromolyn/nedocromil.

46. The method of claim 45, wherein the frequency of asthma exacerbations is assessed according to an annualized rate of severe asthma exacerbations (AAER).

47. The method of claim 45, wherein the frequency of asthma exacerbations is assessed according to a time between subsequent severe asthma exacerbations.
